(19) United States
(12) Patent Application Publication
(10) Pub. No.: US 2002/0177166 A1
(43) Pub. Date: Nov. 28, 2002

(54) Binding motif of a receptor

(75) Inventors: Mark Andrew Guthridge, Kent Town (AU); Michael Claude Berndt, Mount Eliza (AU); Frank Charles Stomski, Croydon Park (AU); Angel Francisco Lopez, Medindie (AU)

Correspondence Address: DARBY & DARBY P.C., 805 Third Avenue, New York, NY, 10022, US

(73) Assignee: MEDVET SCIENCE PTY.LTD.

(21) Appl. No.: 10/099,895

(22) Filed: Mar. 14, 2002

Related U.S. Application Data
(63) Continuation of application No. PCT/AU00/01119, filed on Sep. 15, 2000.

(30) Foreign Application Priority Data
Sep. 12, 2000 (AU) PQ 8733

(51) Int. Cl. C07K 14/715; G01N 33/53; C07H 21/04; C12P 21/02; C12N 5/06

(52) U.S. Cl. 435/7.1; 435/69.1; 435/320.1; 435/325; 530/350; 536/23.5

(57) ABSTRACT

The present invention relates to a binding motif of a receptor. In particular, the binding motif is a cytoplasmic domain associated with stimulation of receptor mediated activities. The present invention further contemplates methods of using the motif in particular for mediating activities of receptors.

In one aspect of the present invention, there is provided a binding motif of a receptor capable of binding a cytoplasmic protein, said binding motif comprising an amino acid sequence wherein at least one amino acid is serine/threonine preferably there are at least two serines to indicate the cytoplasmic binding motif.

Preferably the binding motif comprises an amino acid sequence including the sequence:

-S-X-S/T-

wherein the X is any amino acid.

### CROSS-REFERENCE TO RELATED APPLICATIONS

This application is a continuation of co-pending International Application No. PCT/AU0/01118 filed September 2000, the entire disclosure of which is incorporated herein by reference.

### FIELD OF THE INVENTION

The present invention relates to a binding motif of a receptor. In particular, the binding motif is a cytoplasmic domain associated with stimulation of receptor mediated activities. The present invention further contemplates methods of using the motif in particular for mediating activities of receptors.

### BACKGROUND OF THE INVENTION

The action of signalling molecules such as cytokines has been poorly understood. It is apparent that these cellular proteins can switch on activities within cells. However, the actual triggering mechanisms and how these are relayed to culminate in their final activities is not known. Cell cycles are clearly involved but the link between the triggering molecule and receptor and actions such as cell survival, proliferation, and differentiation is unclear.

Proteins including human granulocyte-macrophage colony-stimulating factor (GM-CSF), interleukin-3 (IL-3), and IL-5 are capable of stimulating normal and transformed hematopoietic cells. With each, the initiating event for signal transduction is the binding of the protein to their surface receptors. These receptors may be composed of subunits such as the α chain and a common β chain (β_{c}) Engagement of β_{c} by the binding of the cytoplasmic protein to surface receptors results in the stimulation of cell survival, proliferation, and differentiation and mature cell effector function in the appropriate lineage, a fact that emphasises the major signaling role played by β_{c} in mediating receptor induced biological activities.

One of the first events in activation of receptors and in the initiation of the signaling cascade is tyrosine phosphorylation of β_{c}. This is a common theme among receptor signaling subunits and can be seen in homodimeric receptors such as the erythropoietin (EPO) receptor, thrombopoietin (TPO) receptor, and granulocyte colony-stimulating factor (G-CSF) receptor as well as in heterodimeric receptors such as in the IL-6 and IL-2 receptors, and in the GM-CSF, IL-3, and IL-5 receptor systems.

Tyrosine phosphorylation of receptor signaling subunits appeared to be a critical step in the creation of docking sites for the association of signaling molecules. Despite the perceived importance of tyrosine phosphorylation of receptors, it is becoming apparent that signaling can proceed in its absence. This is demonstrated in the EPO and TPO receptors, in which the substitution of all tyrosines failed to abolish their activities.

It has been unclear until now how the binding of proteins to their receptors can result in the specialised functions associated with these molecules and their receptors. The signaling events which lead to the specialised functions are unknown. However various cellular proteins are implicated in the cascade of events culminating in the biological functions associated with various molecules. There are many ubiquitous proteins involved in cell signaling pathways and any one or more may be involved in relaying signals switched on by proteins binding to their receptor.

The 14-3-3 family of proteins is one such protein, which consists of 7 different isoforms and is expressed ubiquitously from yeast to humans. The ability of 14-33 to bind to a number of motifs in a wide range of signaling molecules suggests that 14-3-3 proteins may participate in a number of cell signaling pathways that may include mitogenesis, transformation and survival. Although 14-3-3 has been shown to bind a number of signaling molecules, it has been more difficult to determine how or where 14-3-3 can regulate signaling events directly or indirectly, or whether 14-3-3 is implicated at all.

Accordingly, an object of the present invention is to overcome some of the problems of the prior art and to understand how proteins can express their biological activities and to use this information to manipulate cellular functions.

### SUMMARY OF THE INVENTION

In one aspect of the present invention, there is provided a binding motif of a receptor capable of binding a cytoplasmic protein, said binding motif comprising an amino acid sequence wherein at least one amino acid is serine/threonine.

Preferably, the serine/threonine residue corresponds to a serine residue at position 585 of the common β_{c} according to FIG. 1.

Preferably the binding motif comprises an amino acid sequence including the sequence

-S-X-S/T-

wherein the X is any amino acid.

Applicants have found that the second serine/threonine of the motif is an indicator of the cytoplasmic protein binding motif. However, the motif as a whole is involved in the cytoplasmic binding and requires the serine/threonine residue along with flanking amino acids.

Preferably, the motif includes flanking amino acid sequences which may improve the binding of a cytoplasmic protein to the binding motif. More preferably the flanking amino acids are selected from R and X-P (wherein X is any amino acid such that the flanking amino acids individually or co-operatively contribute to the binding motif for binding to a cytoplasmic protein.

More preferably the amino acid sequence of the binding motif includes the sequence:

-R-S-X-S/T-X-P-

wherein X is any amino acid.

In a preferred aspect of the present invention, there is provided a binding motif of a receptor capable of binding a cytoplasmic protein, said binding motif comprising an amino acid sequence, a functional equivalent or analogue thereof and wherein at least two (2) of the amino acids are serine.

The receptor may be any receptor that is capable of binding to an extracellular molecule or protein and which mediates its function through the binding of a cytoplasmic molecule or protein such as 14-3-3 protein, or any cytoplasmic molecule or protein capable of binding a further signaling molecule which activates a cascade of events leading to cell signaling pathways and biological functions such as mitogenesis, proliferation, transformation, differentiation and cell survival, or any other cytoplasmic molecule or protein which does not signal. This molecule may act as an antagonist. More preferably, the cytoplasmic protein is 14-3-3.

Preferably the receptor is selected from the group including:

(1) GM-CSF/IL-3/IL-5 receptor

(2) IL6 human interleukin-6 receptor beta chain precursor (IL-6R-beta)

(3) LEPR human leptin receptor precursor (LEP-R) (OB RECEPTOR) (OB-R).

(4) TNR2 human tumor necrosis factor receptor 2 precursor (tumor necrosis factor

(5) VGR1 human vascular endothelial growth factor receptor 1 precursor

(6) TRK3 human receptor protein-tyrosine kinase TKT precursor (EC 2.7.1.112)

(7) Q01974 protein-tyrosine kinase transmembrane receptor ROR2 precursor

(8) FGR1 human basic fibroblast growth factor receptor 1 precursor (BFGF-R)

(9) Q15426 protein-tyrosine phosphatase, receptor-type, H precursor (EC 3.1.3.48)

(10) PTPM human protein-tyrosine phosphatase mu precursor (EC 3.1.3.48) (R-PTP-MU).

(11) PGDS human alpha platelet-derived growth factor receptor precursor (EC 2.7.1.112)

(12) FGR4 human fibroblast growth factor receptor 4 precursor (FGFR-4) (EC 2.7.1.112)

(13) FGR2 human fibroblast growth factor receptor 2 precursor (FGFR-2) (EC 2.7.1.112)

(14) Q13635 patched protein homolog (PTC)

(15) MANR human macrophage mannose receptor precursor.

(16) LRP2 human low-density lipoprotein receptor-related protein 2 precursor (megalin)

(17) IDD human integral membrane protein dgcr2/idd precursor (KIAA0163)

(18) AMFR human autocrine motility factor receptor precursor (AMF receptor) (gp78)

(19) ACH5 human neuronal acetylcholine receptor protein, alpha-5 chain precursor.

(20) KKIT human: stem cell growth factor receptor (proto-oncogene tyrosine-protein kinase kit) (C-KIT) (CD1117 antigen)

(21) TPOR human: thrombopoietin receptor precusor (TPO-R) (myeloproliferative leukemia protein (C-MPL). (TPOR or MPL).

(22) TPOR mouse: thrombopoietin receptor precursor (TPO-R) (myeloproliferative leukemia protein) (C-MPL). (TPOR or MPL).

Preferably, the binding motif of the receptor is capable of interacting with a cytoplasmic molecule or protein such as 14-3-3 protein, or any cytoplasmic molecule or protein capable of binding a further signaling molecule which activates a cascade of events leading to cell signaling pathways and biological functions such as mitogenesis, proliferation, transformation, differentiation and cell survival, or any other cytoplasmic molecule or protein which does not signal. This molecule may act as an antagonist. More preferably, the cytoplasmic protein is 14-3-3.

Preferably the binding motif of a receptor has an amino acid sequence selected from the group including:

(i) HSRSLP

(ii) SSSRP

(iii) SNSKP

(iv) SDSSP

(v) SISAP

(vi) SLSLP

(vii) SASTP

(viii) SPSFP

(ix) SNSQP

(x) SVSSP

(xi) STSVP

(xii) SKSPP

(xiii) SRSQP

(xiv) SSSLP

(xv) SSSGP

(xvi) SSSFP

(xvii) SPSYP

(xviii) SGSLP

(xix) SQSSP

(xx) SPSLP

(xxi) SGSTP

(xxii) SVSPP

(xxiii) SGSGP

(xxiv) SLGSSP

(xxv) SSSQP

(xxvi) KSSERTP

(xxvii) KSSESTP

or a functional equivalent or analogue thereof.

In another aspect of the present invention there is provide a phosphorylated binding motif of a receptor capable of binding a cytoplasmic protein, said binding motif comprising an amino acid sequence wherein at least one amino acid is serine.

Preferably, the serine residue corresponds to a serine residue at position 585 of the common β_{c} according to FIG. 1.

In a preferred aspect of the present invention, there is provided a phosphorylated binding motif of a receptor capable of binding a cytoplasmic protein, said binding motif comprising an amino acid sequence, a functional equivalent or analogue thereof and wherein at least two (2) of the amino acids are serine and wherein at least one serine residue of the motif is phosphorylated.

In a further preferred aspect, thereis provided a phophorylated binding motif of a GM-CSF/IL-3/IL-5 receptor capable of binding a cytoplasmic protein, said binding motif comprising an amino acid sequence of at least ⁵⁸²HSRSLP⁵⁸⁷ of the GM-CSF/IL-3/IL-5 receptor or a functional equivalent or analogue thereof wherein at least Ser⁵⁸⁵ is phosphorylated.

In a further preferred aspect, there is provided a binding motif of a GM-CSF/IL3/IL-5 receptor capable of binding a cytoplasmic protein, said binding motif comprising an amino acid sequence of at least ⁵⁸²HSRSLP⁵⁸⁷ of the GM-CSF/IL3/IL-5 receptor or a functional equivalent or analogue thereof wherein at least Ser⁵⁸⁵ is phosphorylated.

In another aspect of the present invention, there is provided a method of binding a cytoplasmic protein to a receptor, said method comprising:

phosphorylating a binding motif of a receptor as described above, a functional equivalent or analogue thereof; and

subjecting the binding motif of the receptor to a cytoplasmic protein.

In another aspect of the present invention, there is provided a method of phosphorylating a binding motif of a receptor capable of binding a cytoplasmic protein, said binding motif comprising an amino acid sequence of, a functional equivalent or analogue thereof and wherein at least one amino acid is serine, said method comprising binding a triggering molecule to the receptor.

Preferably, the serine residue corresponds to a serine residue at position 585 of the common β_{c} according to FIG. 1.

In another aspect of the present invention, there is provided a method of phosphorylating a binding motif of a receptor capable of binding a cytoplasmic protein, said binding motif comprising an amino acid sequence, a functional equivalent or analogue thereof and wherein at least two (2) amino acids are serine, said method comprising binding a triggering molecule to the receptor.

In another aspect of the present invention, there is provided a method of activating cellular activities said method including:

regulating the activation of phosphorylation of a binding motif of a receptor as described above, a functional equivalent or analogue thereof; and

subjecting the binding motif to a cytoplasmic protein, wherein said cytoplasmic protein is associated with activation of cellular activities.

In yet another aspect of the present invention there is provided a method of regulating cellular activities, said method including:

regulating the activation of phosphoryaltion of a binding motif of a receptor as described above, a functional equivalent or analogue thereof; and

subjecting the binding motif to a cytoplasmic protein to bind the cytoplasmic protein to the binding motif; and

activating a cell signaling pathway by interacting the bound cytoplasmic protein with a signaling molecule involved in the pathway.

### IN THE FIGURES

FIG. 1 shows the amino acid sequence of the common β chain (β_{c}).

FIG. 2 shows the amino acid sequence of the Stem Cell Growth Factor Receptor (Proto-Oncogene Tyrosine-Protein Kinase Kit) (C-KIT) (CD 117 Antigen)

FIG. 3 shows the amino acid sequence of the Human Thrombopoietin Receptor Precursor (TPO-R) (Myeloproliferative Leukaemia Protein) (C-MPL).

FIG. 4 shows the amino acid sequence of the Mouse Thrombopoietin Receptor Precursor (TPO-R). (Myeloproliferative Leukaemia Protein) (C-MPL).

FIG. 5 shows that Human β_{c} associates with 14-3-3ζ and this association is mediated by the 544-626 region of β_{c} HEK 293T cells were either mock transfected (mock), transfected with wild type β_{c} (wt) or β_{c} containing C terminal deletions. Lysates were prepared from transfected cells and either immunoprecipitated with anti-14-3-3ζ antibody (A) or precipitated with either 14-3-3-GST sepharose (B) or GST-sepharose (C). All proteins were separated on 7.5% SDS-PAGE under reducing conditions before Western blotting with anti-β_{c} antibody (MAb 1C1).

FIG. 6 shows that 14-3-3ζ specifically binds the HSRSLP motif of the β_{c} cytoplasmic domain. (A) HEK 293 cells were used either untransfected (UT), or transfected with wild type β_{c} (wt), or with β_{c} containing the sequence ⁵⁸¹PHSRSLP⁵⁸⁷ mutated to ⁵⁸¹GEFAAAA⁵⁸⁷ or with β_{c} containing the sequence ⁸²⁰RSKPSSP⁸²⁶ mutated to ⁸²⁰EFAAAAA⁸²⁶. Lysates were made and immunoprecipitations were performed using GST-14-3-3-sepharose. The presence of β_{c} was determined by Western blotting with an anti-β_{c} antibody (MAb 1C1). (B) The level of expression β_{c} in the lysates was determined by Western blotting with an anti-β_{c} antibody (MAb 1C1).

FIG. 7 shows that Inhibition of β_{c} association with 14-3-3ζ by phosphorylated but not by unphosphorylated β_{c} and raf-1 peptides. Lysates of HEK293T cells transfected with β_{c} were immunoprecipitated with GST-14-3-3-sepharose in the absence or in the presence of chemically-synthesised peptides (100 uM) containing a β_{c} sequence (CLGPPHSRSLPDILG) or a Raf-1 sequence (CLSQRQRSTSTPNVHM). In the phosphorylated peptides the relevant phosphorylated serine is underlined. The experiment was performed on 7.5% SDS-PAGE under reducing conditions. The presence of β_{c} in the precipitation experiment was determined by Western blotting with anti-β_{c} antibody (MAb 1C1).

FIG. 8 shows that Specific inhibition of β_{c} association with 14-3-3ζ by a phosphorylated peptide encompassing the 579-592 region of β_{c}. Lysates of HEK293T cells transfected with β_{c} were precipitated with GST-14-3-3-sepharose in the absence or in the presence of various concentrations of chemically-synthesised peptides. Two β_{c} peptide sequences were used, CLGPPHSRSLPDILG either non phosphorylated (A) or serine⁵⁸⁵ phosphorylated (B), and CPLSLRSKPSPGPGP either non phosphorylated (C) or serine phosphorylated (D). The appropriate phosphorylated serine in each peptide is underlined. The experiment was performed on 7.5% SDS-PAGE under reducing conditions. The presence of β_{c} in the immunoprecipitates was determined by Western blotting with anti-β_{c} antibody (MAb 1C1).

FIG. 9 shows that Binding of ¹²⁵I-labeled 14-3-3z to synthetic peptides corresponding to the 14-3-3 binding region of β_{c}. Microtiter wells were coated with synthetic peptides CLGPPHSRSLPDILG either non phosphorylated or phosphorylated on the second serine (underlined). Various concentrations of 125I-labeled recombinant 14-3-3z protein were added to microtiter wells and incubated at 22° C. for 2 h. [Insert: Scatchard analyses of 14-3-3 interaction with the serine-phosphorylated peptide].

FIG. 10 shows that ⁵⁸⁵Ser in β_{c} is phosphorylated in vivo by GM-CSF. (A) The anti-phospho-⁵⁸⁵Serβ_{c} antibody specifically recognises the phosphorylated CLGPPHSRSLDILG peptide. Dot blots were prepared on nitrocellulose filters of either the non phosphorylated or the serine phosphorylated CLGPPHSRSLPDILG peptide and the scrambled peptide CLPLSGPDSHIRGPL before probing with anti-phospho-⁵⁸⁵Serβ_{c} antibody. (B) The serine phosphorylated CLGPPHSRSLPDILG peptide specifically inhibits the binding of anti-phospho-⁵⁸⁵Serβ_{c} antibody to β_{c}. Lysates of HEK293T cells transfected with wild type β_{c} were immunoprecipitated with anti-β_{c} antibody (MAb 8E4). Immunoprecipitates were run on 7.5% SDS-PAGE under reducing conditions. Anti-phospho-⁵⁸⁵Serβ_{c} antibody was then pre-incubated with either medium (none), 100 fold molar excess of the serine phosphorylated (1) or non phosphorylated (2) CLGPPHSRSLPDILG peptide, or the scrambled peptide CLPLSGPDSHIRGPL (3). The filters were then Western blotted and probed with the pretreated anti-phospho-⁵⁸⁵Serβ_{c} antibody. (C) Upregulation of ⁵⁸⁵Ser phosphorylation by GM-CSF. M1 cells expressing GM-CSFRa and β_{c} were either untreated (−) or stimulated with GM-CSF (2 ng/ml) for 30 seconds (+). Lysates of the M1 cells were immunoprecipitated with anti-β_{c} antibody (MAb 8E4) and the immunoprecipitates run on 10% SDS-PAGE under reducing conditions. The filters were then Western blotted with either anti-phospho-⁵⁸⁵Serβ_{c} antibody or the anti-β_{c} antibody (MAb 1C1).

FIG. 11 shows Phosphorylation of Ser585 of β_{c} in response to IL-3 and the recruitment of 14-3-3. CTL-EN cells expressing IL-3Ra and either wtβ_{c} or β_{c}HSRSLP→EFAAAA were washed, starved for 4 hours in DMEM containing 0.1% FCS and then stimulated with 50 ng/ml IL-3 for the indicated times. Cells were then lysed and the β_{c} immunoprecipitated (7×10⁷ cells/IP) with the 1C1 anti-β_{c} specific mAb. Immunoprecipitates were divided and 80% or 20% were separately run on SDS-PAGE and transferred to nitrocellulose filters. (a) 80% of the immunoprecipitates were probed with the anti-phospho-Ser585β_{c} antibody, the filters stripped, and reprobed with the 1C1 anti-β_{c} mAb. 20% or the immunoprecipitates were probed with the 4G10 anti-phosphotyrosine antibody. (b) Regulation of 14-3-3 association with β_{c} following IL-3 stimulation. CTL-EN cells were starved and stimulated as above, and β_{c} was immunoprecipitated with the 1C1 mAb coupled to Sepharose beads. Immunoprecipitates were boiled in a non-reducing sample buffer and subjected to SDS-PAGE on a non-reducing gel. Immunoblot analysis was performed with anti-14-3-3 antibodies and the filter stripped and re-probed with the 1C1 antibody. These results were typical of 3 experiments.

FIG. 12 shows PKA phosphorylates Ser585 of β_{c}. a) The ability of either PKA or CKII to phosphorylate purified hisβ_{c}445-881 was examined in an in vitro kinase assay using g³²PATP. Peptide competitions were also performed using 200 mM peptides containing the indicated sequences. b) The ability of PKA and CKII to phosphorylate Ser585 of hisβ_{c}445-881 was examined by immunoblot analysis using the anti-phospho-Ser585β_{c} antibodies. Filters were probed firstly with anti-phospho-Ser585β_{c} antibodies (top panel). Filters were then stripped and reprobed with an antibody that recognizes the HSRSLP motif of PC regardless of whether Ser585 is phosphorylated or not (anti-HSRSLPβ_{c})(bottom panel). c) To determine if agents that eleveate intracellular cAMP and activate PKA also resulted in increased phosphorylation of β_{c}Ser585, cells were starved for 12 hours and left unstimulated (nil), or stimulated with IL-3 (50 ng/ml), forskolin (50 mM) or dibutyryl cAMP (100 mM) for 10 minutes at 37° C. Cells were then lysed and β_{c} immunoprecipitated with 1C1. Ser585 phosphorylation of β_{c} was determined using the anti-phospho-Ser585 antibodies (top panel). The filter was then stripped and re-probed with anti β_{c} 1C1 antibodies.

FIG. 13 shows 14-3-3 binding to β_{c} in response to IL-3 stimulation is required for the recruitment and activation of PI 3-kinase. (a) analysis of PI 3-kinase activity. CTL-EN cells expressing IL-3Ra and either wtβ_{c} or β_{c}HSRSLP→EFAAAA were starved for 12 hours in DMEM containing 0.5% FCS and stimulated with 50 ng/ml IL-3 for the indicated times. Cells were then lysed and phospho-tyrosine containing proteins were immunoprecipitated (2×10⁷ cells/IP) using the 4G10 antibody. Immunoprecipitates were washed in 1× PI 3-kinase buffer (20 mM HEPES, pH 7.5, 5 mM MgCl₂, 1 mM EGTA) and PI 3-kinase activity was analysed in a lipid kinase assay using phosphatidyl inositol and g³²PATP as substrates as described in the “Materials and Methods”. Autoradiogram of TLC plate is shown with ³²P-labelled phosphatidyl inositols (PIP) and the origin indicated. These results were typical of 3 experiments. (b) CTL-EN cells were also examined for the association of β_{c} with p85. CTL-EN cells expressing either wtβ_{c} or β_{c}HSRSLP→EFAAAA were starved for 12 hours in DMEM containing 0.5% FCS and then stimulated with 50 ng/ml IL-3 for the indicated times. Cells were then lysed and the β_{c} immunoprecipitated (2×10⁷ cells/IP) with the 1C1 anti-β_{c} specific mAb. Immunoprecipitates were then subjected to SDS-PAGE, transferred to nitrocellulose and immunoblotted with anti-p85 (1 mg/ml) followed by detection with HRP-conjugated anti-rabbit IgG antibody and ECL. The filters were then stripped, probed with anti-SHP2 (1 mg/ml), stripped again and finally probed with 1C1 anti-β_{c} (2 mg/ml). These results were typical of 2 experiments. The association of hisβ_{c}445-881 with p85 was also assessed in pull-down experiments together with peptide competition. (c) List of peptides used in the hisbβ_{c}445-881 pull-down experiment. S refers to a phospho-serine residue. (d) Association of p85 with recombinant hisβ_{c}445-881. hisβ_{c}445-881 (10 mg) coupled to Sepharose resin and phosphorylated on Ser585 with PKA (lanes 1-5) or Sepharose alone (lane 6) was incubated with COS-7 pre-cleared lysates in the absence (lane 1) or in the presence of 200 mM competing peptides (lane 2, scrambled. Lane 3, Ser585Ala. Lane 4, non-phospho-Ser585. lane 5, phospho-Ser585) for 1 hour at 4° C. The resin was then washed extensively, subjected to SDS-PAGE and transferred to a nitrocellulose filter. The filter was immunoblotted with anti-p85 antibodies (1 mg/ml) followed by detection with HRP-conjugated anti-rabbit IgG antibody and ECL. These results were typical of 2 experiments.

FIG. 14 shows 14-3-3 binding to β_{c} is required for Akt activation but not STAT5, ERK or JNK activation. (a) CTL-EN cells expressing IL-3Ra and either wtβ_{c} or β_{c}HSRSLP→EFAAAA were starved for 12 hours in DMEM containing 0.5% FCS and then stimulated with 50 ng/ml IL-3 for the indicated times. Cells were lysed, whole cell lysates were subjected to SDS-PAGE and transferred to a nitrocellulose filter. Filters were immunoblotted and stripped sequentially with anti-phospho Akt pAb (1:500) anti-phosphorylated STAT5 mAb (2 mg/ml), anti-active MAPK pAb (50 ng/ml) and anti-ERK pAb (1 mg/ml) followed by detection with HRP-conjugated anti-rabbit or anti-mouse IgG antibody and ECL. (b) CTL-EN cells expressing IL-3Ra and either wtβ_{c} or β_{c}HSRSLP→EFAAAA were starved for 12 hours in DMEM containing 0.5% FCS and stimulated with 50 ng/ml IL-3 for the indicated times. Cells were lysed and JNK was immunoprecipitated. The immunoprecipitates were then washed in 1× JNK buffer (10 mM MgCl₂, 10 mM Tris-HCl ph 7.4) and the JNK activity was then analysed in an in vitro kinase assay using g³²PATP and 1 mg GST-jun1-79. Kinase assays were subjected to SDS-PAGE and transferred to a nitrocellulose filter. An autoradiogram of the filters is shown.

FIG. 15 shows 14-3-3 binding to β_{c} in response to IL-3 regulates cellular viability. (a) CTL-EN cells expressing IL-3Ra and either wtβ_{c} or β_{c}HSRSLP→EFAAAA were washed and then plated in duplicate at 2.5×10⁵ cells/ml in DMEM containing 0.1% FCS and either 10 ng/ml IL-3, 10 ng/ml IL-2 or long/ml IL-3 and 5 mM LY294002. Viable cells were counted using the trypan blue exclusion method after 4 days. The results shown are typical of 3 experiments. (b) CTL-EN cells expressing IL-3Ra and either wtβ_{c} or β_{c}HSRSLP→EFAAAA were washed and then plated in triplicate at 5×10⁵ cells/ml in DMEM containing 0.1% FCS and either no IL-3, 1 ng/ml IL-3 or 10 ng/ml IL-3. Metabolic activity was measured each day using the CellTiter96 AQᵤₑₒᵤₛ one solution cell proliferation assay according to the manufacturers instructions. Shown are the raw absorbance values (490 nm) plotted against day for CTL-EN cells expressing wtβ_{c} in the presence of no IL-3 (ᵤ), 1 ng/ml IL-3 (I) or 10 ng/ml IL3 (n) and CTL-EN cells expressing the β_{c}HSRSLP→EFAAAA mutant in the presence of no IL-3 (u) or 10 ng/ml IL-3 (ₜ). These results are typical to 2 experiments.

FIG. 16 shows 14-3-3 binding to β_{c} is not required for cell cycle progression. a) CTL-EN cells expressing IL-3Ra and either wtβ_{c} or β_{c}HSRSLP→EFAAAA were washed and then plated at 2.5×10⁵ cells/ml in DMEM containing 0.1% FCS and starved for 24 hours. Cells were then stimulated for a further 24 hours with 50 ng/ml IL-3 in the same medium after which the cells were harvested by centrifugation, fixed in ethanol and stained with propidium iodide. Cell cycle distribution was then analysed by flow cytometry. b) c-myc induction in response to IL-3 stimulation. CTL-EN cells were washed, starved and stimulated with IL-3 for 2 hours as described above. Total RNA was extracted and subjected to Northern blot analysis. Filters were probed sequentially with ³²P-labelled cDNAs for c-myc and 18SrRNA. Signals were detected by autoradiography.

FIG. 17 shows 14-3-3 binding to β_{c} in response to IL-3 stimulation promotes cellular survival by suppressing apoptosis. CTL-EN cells expressing IL-3Ra and either wtβ_{c} or β_{c}HSRSLP→EFAAAA were washed and then plated at 5×10⁵ cells/ml in DMEM containing 0.1% FCS and either long/ml IL-3 or 10 ng/ml IL-2 for 18 hours. Cells were then harvested by centrifugation and stained with annexin V according to the manufacturers instructions. Annexin V positive cells were then analysed by flow cytometry.

FIG. 18 shows a proposed model for the regulation of survival by IL-3. Binding of IL-3 to the IL-3 receptor, composed of an α-chain (α) and a 62 -Chain (β_{c}), results in receptor oligomerization (only one α-chain and one β-chain are shown for simplicity), increased tyrosine phosphorylation and also increased Ser585 phosphorylation (S). Ser585 phosphorylation of β_{c} allows recruitment of 14-3-3, which in tun recruits PI 3-kinase (PI 3-K) either directly, or indirectly through an additional dapto molecule. The recruitment of PI 3-kinase and the production of phosphatidyl inositols (PIP) couples the activated receptor to down-stream signalling molecules such as Akt and promotes cellular survival.

FIG. 19 shows ⁵⁸⁵Ser in β_{c} of acute myeloid leukaemia (AML) is constitutively phosphorylated. CTL-EN cells expressing IL-3Rα and β_{c} were either untreated or stimulated with IL-3 (2 ng/ml) for 1 or 5 minutes. AML cells expressing GM-CSFRα and β_{c} were either untreated or stimulated with GM-CSF (2 ng/ml) for 5 minutes. Lysates of either CTL-EN cells or AML cells were immunoprecipitated with anti-β_{c} antibody (MAb 8E4) and the immunoprecipitates run on 10% SDS-PAGE under reducing conditions. The filters were then western blotted with either anti-phospho-⁵⁸⁵Serβ_{c} antibody (A) or the anti-β_{c} antibody (MAb 1C1) (B).

FIG. 20 shows phosphorylation of Ser585 of β_{c} in response to IL-3 and the recruitment of 14-3-3.

FIG. 21 shows PKA phosphorylates Ser585 of β_{c}. (A) The ability of either PKA or CKII to phosphorylate purified Hisβ_{c}445-881 was examined in an in vitro kinase assay using γ³² P-ATP. (B) The ability of PKA and CKII to phosphorylate Ser585 of Hisβ_{c}445-881 was examined by immunoblot analysis using the anti-phospho-Ser585β_{c} antibody. (C) Determination of the Kₘ and the Vₘₐₓ for PKA phosphorylation of the β_{c} peptide. (D) Pharmacology regulation of PKA activity and its effect on β_{c}Ser585 phosphorylation.

FIG. 22 shows 14-3-3 binding to β_{c} in response to IL-3 stimulation is required for the recruitment and activation of PI 3-kinase. (A) Analysis of PI 3-kinase activity. (B) CTL-EN cells were also examined for the association of β_{c} with p85. (C) The association of recombinant Hisβ_{c}445-881 with p85 and 14-3-3.

FIG. 23 shows 14-3-3 binding to β_{c} is required for Akt activation but not STAT5, ERK, JNK or JAK2 activation. (A) Phosphorylation of Akt, STAT5 and ERK2. (B) Activation of JNK activity. (C) Tyrosine phosphorylation of JAK2.

FIG. 24 shows 14-3-3 binding to β_{c} in response to IL-3 stimulation promotes cellular survival by suppressing apoptosis. (A) Cellular viability. Shown is the viability of CTL-EN cells expressing wtβ_{c} in the presence of IL-2 (●) or IL-3 (▪) and the viability of CTL-EN cells expressing β_{c}HSRSLP→EFAAAA in the presence of IL-2 (▾) or IL-3 (▴). Shown are the raw absorbance values (490 nm) plotted against day for CTL-EN cells expressing wtβ_{c} in the presence of no IL-3 (▾), or long/ml IL-3 (▪), and CTL-EN cells expressing the β_{c}HSRSLP EFAAAA mutant in the presence of no IL-3 (♦), or 10 ng/ml IL-3 (▴). (C) DNA laddering. (D) Annexin V and propidium iodide staining.

FIG. 25 shows 14-3-3 binding to β_{c} is not required for cell cycle progression. (A) The distribution of cells in G₀/G₁, S and G₂/M phases. (B) c-myc induction in response to IL-3 stimulation.

FIG. 26 shows proposed model for the regulation of survival by IL-3.

FIG. 27 shows Ser585 phosphorylation and PI 3-K signalling is constitutive in primary human AML cells.

### DETAILED DESCRIPTION OF THE INVENTION

In one aspect of the present invention, there is provided a binding motif of a receptor capable of binding a cytoplasmic protein, said binding motif comprising an amino acid sequence wherein at least one amino acid is serine/threonine.

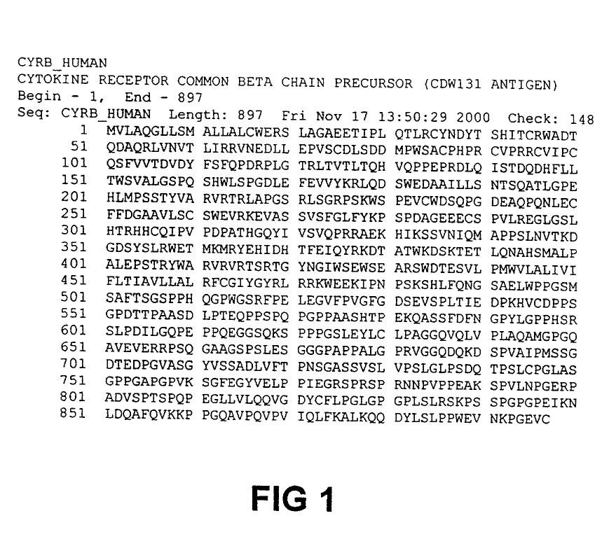

Preferably, the serine/threonine residue corresponds to a serine residue at position 585 of the common β_{c} according to FIG. 1.

In a preferred aspect of the present invention, there is provided a binding motif of a receptor capable of binding a cytoplasmic protein, said binding motif comprising an amino acid sequence, a functional equivalent or analogue thereof and wherein at least two (2) of the amino acids are serine.

Preferably the binding motif comprises an amino acid sequence including the sequence:

-S-X-S/T-

wherein the X is any amino acid.

Applicants have found that the second serine/threonine of the motif is an indicator of the cytoplasmic protein binding motif. However, the motif as a whole is involved in the cytoplasmic binding and requires the serine/threonine residue along with flanking amino acids.

Preferably, the motif includes flanking amino acid sequences which may improve the binding of a cytoplasmic protein to the binding motif. More preferably the flanking amino acids are selected from R and X-P (wherein X is any amino acid such that the flanking amino acids individually or co-operatively contribute to the binding motif for binding to a cytoplasmic protein.

More preferably the amino acid sequence of the binding motif includes the sequence:

-R-S-X-S/T-X-P-

wherein X is any amino acid.

The term “functional equivalent or analogue thereof” as used herein means a sequence which functions in a similar way but may have deletions, additions or substitutions that do not substantially change the activity or function of the sequence.

The term “comprising” or “comprises” or variations of the word as used herein is not intended to exclude other additives, components integers or steps.

The term “motif” as used herein in relation to the receptor, means a distinctive portion of the receptor but is not intended to include the whole receptor.

The receptor may be any receptor that is capable of binding to an extracellular molecule or protein and which mediates its function through the binding of a cytoplasmic molecule or protein such as 14-3-3 protein, or any cytoplasmic molecule or protein capable of binding a further signaling molecule which activates a cascade of events leading to cell signaling pathways and biological functions such as mitogenesis, proliferation, transformation, differentiation and cell survival, or any other cytoplasmic molecule or protein which does not signal. This molecule may act as an antagonist. More preferably, the cytoplasmic protein is 14-3-3.

Signaling molecules may be molecules involved in cellular pathways such as those pathways involved in proliferation, survival or differentiation. Examples of such pathways may include the JAK/STAT pathway, the ras/MAP kinase pathway or the PI-3-Kinase pathway. All pathways may be involved directly or indirectly with these functions.

Preferably the receptor is selected from the group including:

(1) GM-CSF/IL-3/IL-5 receptor

(2) IL6 human interleukin-6 receptor beta chain precursor (IL-6R-beta)

(3) LEPR human leptin receptor precursor (LEP-R) (OB RECEPTOR) (OB-R).

(4) TNR2 human tumor necrosis factor receptor 2 precursor (tumor necrosis factor

(5) VGR1 human vascular endothelial growth factor receptor 1 precursor

(6) TRK3 human receptor protein-tyrosine kinase TKT precursor (EC 2.7.1.112)

(7) Q01974 protein-tyrosine kinase transmembrane receptor ROR2 precursor

(8) FGR1 human basic fibroblast growth factor receptor 1 precursor (BFGF-R)

(9) Q15426 protein-tyrosine phosphatase, receptor-type, H precursor (EC 3.1.3.48)

(10) PTPM human protein-tyrosine phosphatase mu precursor (EC 3.1.3.48) (R-PTP-MU).

(11) PDGS human alpha platelet-derived growth factor receptor precursor (EC 2.7.1.112)

(12) FGR4 human fibroblast growth factor receptor 4 precursor (FGFR-4) (EC 2.7.1.112)

(13) FGR2 human fibroblast growth factor receptor 2 precursor (FGFR-2) (EC 2.7.1.112)

(14) Q13635 patched protein homolog (PTC)

(15) MANR human macrophage mannose receptor precursor.

(16) LRP2 human low-density lipoprotein receptor-related protein 2 precursor (megalin)

(17) IDD human integral membrane protein dgcr2/idd precursor (KIAA0163)

(18) AMFR human autocrine motility factor receptor precursor (AMF receptor) (gp78)

(19) ACH5 human neuronal acetylcholine receptor protein, alpha-5 chain precursor.

(20) KKIT human: stem cell growth factor receptor (proto-oncogene tyrosine-protein kinase kit) (C-KIT) (CD117 antigen)

(21) TPOR human: thrombopoietin receptor precusor (TPO-R) (myeloproliferative leukemia protein (C-MPL). TPOR or MPL.

(22) TPOR mouse: thrombopoietin receptor precursor (TPO-R) (myeloproliferative leukemia protein) (C-MPL). TPOR or MPL.

Preferably, the binding motif of the receptor is capable of interacting with a cytoplasmic molecule or protein, or any cytoplasmic molecule or protein capable of binding a further signaling molecule which activates a cascade of events leading to cell signaling pathways and biological functions such as mitogenesis, proliferation, transformation, differentiation and cell survival, or any other cytoplasmic molecule or protein which does not signal. This molecule may act as an antagonist. More preferably, the cytoplasmic protein is 14-3-3.

The 14-3-3 protein is a family of proteins which consists of 7 different isoforms and is expressed ubiquitously from yeast to humans. The ability of 14-3-3 to bind to a number of motifs in a wide range of signaling molecules suggests that 14-3-3 proteins may participate in a number of cell signaling pathways that may include mitogenesis, transformation and survival. Although 14-3-3 has been shown to bind a number of signaling molecules, it has been more difficult to determine how 14-3-3 can regulate signaling events.

Preferably the binding motif of a receptor has an amino acid sequence selected from the group including:

(I) HSRSLP

(ii) SSSRP

(iii) SNSKP

(iv) SDSSP

(v) SISAP

(vi) SLSLP

(vii) SASTP

(viii) SPSFP

(ix) SNSQP

(x) SVSSP

(xi) STSVP

(xii) SKSPP

(xiii) SRSQP

(xiv) SSSLP

(xv) SSSGP

(xvi) SSSFP

(xvii) SPSYP

(xviii) SGSLP

(xix) SQSSP

(xx) SPSLP

(xxi) SGSTP

(xxii) SVSPP

(xxiii) SGSGP

(xxiv) SLGSSP

(xxv) SSSQP

(xxvi) KSSERTP

(xxvii) KSSESTP

or a functional equivalent or analogue thereof.

The binding capacity of the motif may be analysed by any binding studies or experiments available to the skilled addressee. Such experiments may include measuring the binding ability of a designated cytoplasmic protein to the binding motif. For instance electrophoretic mobility shift assays (EMSA or band shift assays) or foot print assays or pull down experiments are available to measure specific binding.

Hence the binding motif can be identified by the presence of a, serine residue preferably in an amino acid sequence as described above, and the ability to bind a designated cytoplasmic protein. The designated cytoplasmic protein may be 14-3-3 protein, or any cytoplasmic protein capable of binding a further signaling molecule which activates a cascade of events leading to cell signaling pathways and biological functions such as mitogenesis, proliferation, transformation, differentiation and cell survival. More preferably, the cytoplasmic protein is 14-3-3.

Preferably, the receptor is the GM-CSF/IL-3/IL-5 receptor which includes the common beta chain (β_{c}). It is found that the cytokines GM-CSF, IL-3 and IL-5 signal their actions through the surface receptor via the β_{c}. Most preferably, the binding motif comprises a sequence which includes amino acids HSRSLP corresponding to amino acids 582 to 587 of the common β_{c} according to FIG. 1 a functional equivalent or analogue thereof.

The common β chain (β_{c}) is a component of a cytokine receptor and is part of a signaling subunit of the receptor. It is thought that the cytokine signals its functions through the β_{c}, initiating events which cascade and culminate in an identifiable biological function such as cell survival, proliferation, differentiation and mature cell effector functions. However, the present invention is not limited to motifs of the β_{c} but includes motifs of receptors having similar sequences to the β_{c} and including a serine/threonine residue.

The region or motif comprising amino acids 582 to 587 of the common β_{c} may include ⁵⁸²HSRSLP⁵⁸⁷ which preferably interacts with a cytoplasmic protein selected from the group including 14-3-3 protein, WW-domain of the prolyl isomerase, Pin1, and the ubiquitin ligase, NEDD4 or any cytoplasmic protein capable of binding a further signaling molecule which activates a cascade of events leading to cell signaling pathways and biological functions such as mitogenesis, proliferation, transformation, differentiation and cell survival. However the present invention is not limited to this sequence but includes other equivalent sequences capable of performing the same function.

Other binding motifs of receptors according to the present invention include amino acid sequences of:

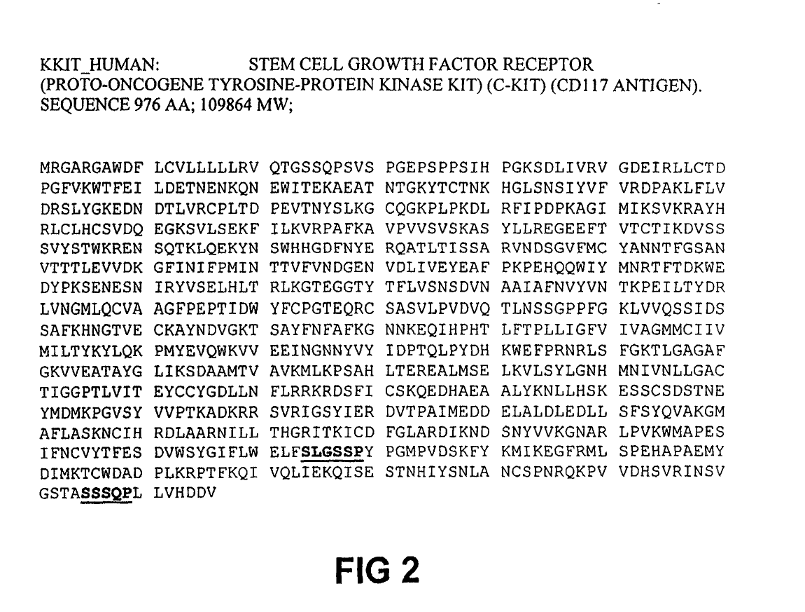

Stem Cell Growth Factor Receptor (C-Kit) (Proto-Oncogene Tyrosine-Protein Kinase Kit) (C-KIT) (CD 117 Antigen), preferably including amino acids 863 to 869 according to FIG. 2 or amino acid residues 965 to 969 according to FIG. 2 or a functional equivalent or analogue thereof.

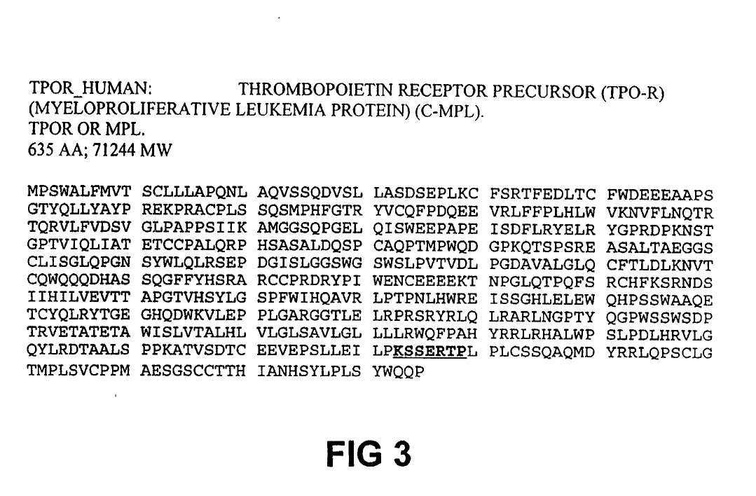

Thrombopoietin Receptor Precursor (TPO-R) (Myeloproliferative Leukemia Protein) (C-MPL). (TPOR or MPL) preferably including amino acids 573 to 579 according to FIG. 3 or a functional equivalent or analogue thereof.

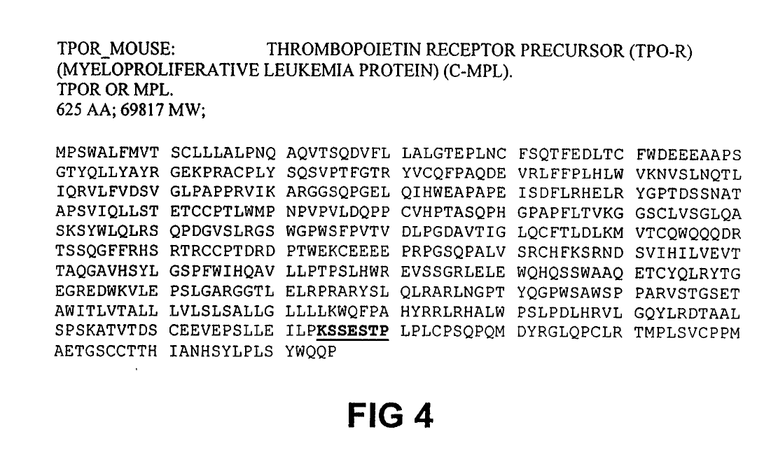

Thrombopoietin Receptor Precursor (TPO-R) (Myeloproliferative Leukemia Protein) (C-MPL). (TPOR or MPL) preferably including amino acids 564 to 570 according to FIG. 4 or a functional equivalent or analogue thereof.

IL6B HUMAN interleukin-6 receptor beta chain precursor (IL-6R-BETA), preferable including amino acids 735-739 having the sequence SSSRP or a functional equivalent or analogue thereof

LEPR HUMAN leptin receptor precursor (LEP-R) (OB receptor) (OB-R), preferably including amino acids 991-995 having the sequence SNSKP or a functional equivalent or analogue thereof.

TNR2 HUMAN tumor necrosis factor receptor 2 precursor (tumor necrosis factor) preferably including amino acids 368-372 having the sequence SDSSP or a functional equivalent or analogue thereof.

VGR1 HUMAN vascular endothelial growth factor receptor 1 precursor, preferably including amino acids 1197-1201 having the sequence SISAP or a functional equivalent or analogue thereof.

TRK3 HUMAN receptor protein-tyrosine kinase TKT precursor (EC 2.7.1.112), preferably including amino acids 444-448, having the sequence SLSLP or a functional equivalent or analogue thereof.

Q01974 protein-tyrosine kinase transmembrane receptor ROR2 precursor, preferably including amino acids 435-439, having the sequence SASTP or a functional equivalent or analogue thereof.

FGR1 HUMAN basic fibroblast growth factor receptor 1 precursor (BFGF-R), preferably including amino acids 777-781, having the sequence SPSFP or a functional equivalent or analogue thereof.

Q15426 protein-tyrosine phosphatase, receptor-type, H precursor (EC 3.1.3.48), preferably including amino acids 1082-1086, having the sequence SNSQP or a functional equivalent or analogue thereof.

PTPM HUMAN protein-tyrosine phosphatase MU precursor (EC 3.1.3.48) (R-PTP-MU), preferably including amino acids 818-822, 833-837, 1082-1086 having the sequences SVSSP, STSVP, SKSPP or functional equivalents or analogues thereof.

PGDS HUMAN alpha platelet-derived growth factor receptor precursor (EC 2.7.1.112), preferably including amino acids 616-620 having the sequence SRSQP or a functional equivalent or analogue thereof.

FGR4 HUMAN fibroblast growth factor receptor 4 precursor (FGFR-4) (EC 2.7.1.112), preferably including amino acids 439-443, 791-795 having the sequences SSSGP, SSSFP or functional equivalents or analogues thereof.

FGR2 HUMAN fibroblast growth factor receptor 2 precursor (FGFR-2) (EC 2.7.1.112), preferably including amino acids 780-784 having the sequence SPSYP or a functional equivalent or analogue thereof.

Q13635 patched protein homolog (PTC), preferably including amino acids 1290-1294 having the sequence SGSLP or a functional equivalent or analogue thereof.

MANR HUMAN macrophage mannose receptor precursor, preferably including amino acids 1432-1436 having the sequence SQSSP or a functional equivalent or analogue thereof.

LRP2 HUMAN low-density lipoprotein receptor-related protein 2 precursor (megalin), preferably including amino acids 4616-4620 having the sequence SPSLP or a functional equivalent or analogue thereof.

IDD HUMAN integral membrane protein DGCR2/IDD precursor (KIAA0163), preferably including amino acids 526-530 having the sequence SGSTP or a functional equivalent or analogue thereof.

AMFR HUMAN autocrine motility factor receptor precursor (AMF receptor) (GP78), preferably including amino acids 203-207 having the sequence SVSPP or a functional equivalent or analogue thereof.

ACH5 HUMAN neuronal acetylcholine receptor protein, alpha-5 chain precursor, preferably including amino acids 382-386 having the sequence SGSGP or a functional equivalent or analogue thereof.

In another aspect of the present invention there is provided a phosphorylated binding motif of a receptor capable of binding a cytoplasmic protein, said binding motif comprising an amino acid sequence wherein at least one amino acid is serine/threonine and wherein the serine residue is phosphorylated.

Preferably, the serine residue corresponds to a serine residue at position 585 of the common β_{c} according to FIG. 1.

In another aspect of the present invention, there is provided a phosphorylated binding motif of a receptor capable of binding a cytoplasmic protein, said binding motif comprising an amino acid sequence, a functional equivalent or analogue thereof and wherein at least two (2) of the amino acids are serine and wherein at least one serine residue of the motif is phosphorylated.

Preferably the binding motif comprises an amino acid sequence including the sequence:

-S-X-S/T-

wherein the X is any amino acid.

Applicants have found that the second serine/threonine motif is an indicator of the cytoplasmic protein binding motif. However, the motif as a whole is involved in the cytoplasmic binding and requires the serine/threonine residue along with flanking amino acids.

Preferably, the motif includes flanking amino acid sequences which may improve the binding of a cytoplasmic protein to the binding motif. More preferably the flanking amino acids are selected from R and X-P (wherein X is any amino acid) such that the flanking amino acids individually or co-operatively contribute to the binding motif for binding to a cytoplasmic protein.

More preferably the amino acid sequence of the binding motif includes the sequence: -R-S-X-S/T-X-P-

wherein X is any amino acid.

The motif must have at least one serine residue Preferably there are two. Preferably, the second serine residue from the 5′ end of the motif is phosphorylated.

Preferably, the receptor is selected from the group of receptors described above and the amino acid sequence is any one of the binding motifs described above.

In a further preferred aspect, there is provided a binding motif of a GM-CSF/IL-3/IL-5 receptor capable of binding a cytoplasmic protein, said binding motif comprising an amino acid sequence including the sequence ⁵⁸²HSRSLP⁵⁸⁷ of the GM-CSF/IL-3/IL-5 receptor or a functional equivalent or analogue thereof wherein at least Ser⁵⁸⁵ is capable of being phosphorylated.

In a further preferred aspect, thereis provided a phophorylated binding motif of a GM-CSF/IL-3/IL-5 receptor capable of binding a cytoplasmic protein, said binding motif comprising an amino acid sequence including the sequence ⁵⁸²HSRSLP⁵⁸⁷ of the GM-CSF/IL-3/IL-5 receptor or a functional equivalent or analogue thereof wherein at least Ser⁵⁸⁵ is phosphorylated.

More preferably, the binding motif binds to the cytoplasmic protein 14-3-3.

It is also preferred that other receptors as described above can be phosphorylated and induced to bind a cytoplasmic protein such as 14-3-3 by phosphorylation preferably of the second serine residue from the 5′ end of the motif.

In another aspect of the present invention, there is provided a method of phosphorylating a binding motif of a receptor capable of binding a cytoplasmic protein, said binding motif comprising an amino acid sequence of, a functional equivalent or analogue thereof and wherein at least one amino acid is serine, said method comprising binding a triggering molecule to the receptor.

Preferably, the serine residue corresponds to a serine residue at position 585 of the common β_{c} according to FIG. 1.

In another aspect of the present invention, there is provided a method of phosphorylating a binding motif of a receptor capable of binding a cytoplasmic protein, said binding motif comprising an amino acid sequence or, a functional equivalent or analogue thereof and wherein at least two (2) of the amino acids are serine, said method comprising binding a triggering molecule to the receptor.

Preferably, the binding motif is any one of the motifs described above.

In a preferred aspect, phosphorylation of the binding motif is caused by the binding of a triggering molecule to its corresponding receptor. Triggering molecules may be cytokines which bind to cytokine receptors. Preferably the receptor is a heterodimeric receptor. More preferably it is a GM-CSF/IL-5/IL-3 receptor bound by a GM-CSF, IL-5 or IL-3 cytokine. Other triggering molecules may be the corresponding triggering molecule that binds to any one of the receptors listed above.

Preferably the binding motif is as described above.

It has been found by the applicants that any triggering molecule which binds to a receptor or receptor signaling system may be capable of causing phosphorylation of the binding motif. Preferably the triggering molecule is a cytokine which binds to a homodimeric or heterodimeric receptor prior to phosphorylation of the binding motif. Preferably the cytokine binds to a heterodimeric cytokine receptor. The heterodimeric cytokine receptor may comprise two (or three) subunits which subserve distinct and specialised functions. These subunits include the major ligand binding subunit (α subunit) and the signaling subunit which may comprise a β or δ subunit. The signaling subunit may recognise several cytokines on the α subunit which can then tranduce signals from the cytokines into the cell.

The common beta chain (β_{c}) is found in the receptor signalling systems of cytokines including granulocyte macrophage colony stimulating factor (GM-CSF), interleukin-3 (IL-3) and interleukin-5 (IL-5). In this system, preferably the binding motif comprises an amino acid sequence including ⁵⁸²HSRSLP⁵⁸⁷ on the β_{c}. More preferably, this sequence is phosphorylated upon binding of GM-CSF, IL-3 or IL-5 to the receptor. More preferably Ser⁵⁸⁵ is phosphorylated particularly when GM-CSF, IL-3 or IL-5 bind to their corresponding receptors.

However, other triggering molecules can cause phosphorylation of an equivalent region of the receptor. For instance, the molecule which binds to the receptor (as listed above) may cause phosphorylation to the binding motif.

In another aspect of the present invention, there is provided a method of binding a cytoplasmic protein to a receptor, said method comprising:

phosphorylating a binding motif of a receptor as described above, a functional equivalent or analogue thereof; and

subjecting the binding motif of the receptor to a cytoplasmic protein.

Although not wishing to be limited by theory, it is perceived that the phosphorylation of the binding motif may improve the binding of a cytoplasmic protein to the binding motif so that when the cytoplasmic protein is reacted with the motif, or equivalent thereof, binding may occur to bring other cytoplasmic proteins or signaling molecules into close proximity to the receptor. Phosphorylation may occur by any means which transfers a phosphoryl (phosphate) group to the cytoplasmic binding motif.

The cytoplasmic protein may be 14-3-3 protein, or any cytoplasmic protein capable of binding a further signaling molecule which activates a cascade of events leading to cell signaling pathways and biological functions such as mitogenesis, proliferation, transformation, differentiation, cell survival, chemotaxis, motility, enhanced phagocytosis, bacterial killing, superoxide production and cytotoxicity.

In another aspect of the present invention, there is provided a method of activating cellular activities said method including:

regulating the activation of phosphorylation of a binding motif of a receptor as described above, a functional equivalent or analogue thereof; and

subjecting the binding motif to a cytoplasmic protein wherein said cytoplasmic protein is associated with cellular activities.

Preferably the cytoplasmic protein is 14-3-3 protein, or any cytoplasmic protein capable of binding a further signaling molecule which activates a cascade of events leading to cell signaling pathways and biological functions such as mitogenesis, proliferation, transformation, differentiation cell survival, chemotaxis, motility, enhanced phagocytosis, bacterial killing, superoxide production and cytotoxicity. Preferably the cytoplasmic binding protein is 14-3-3.

The 14-3-3 molecule binds not only to the cytoplasmic binding motif (as found by the applicants) but has the ability to bind to a wide range of signaling molecules and to participate in a number of cell signaling pathways resulting in mitogenesis, transformation, differentiation cell survival, chemotaxis, motility, enhanced phagocytosis, bacterial killing, superoxide production and cytotoxicity.

Once 14-3-3 or an equivalent binds to the binding motif of the receptor, its ubiquitous nature can bind cytoplasmic proteins involved in signaling pathways which activate these pathways. For instance, not being limited by theory and by example, it has now been found by the Applicants that cytokines such as GM-CSF, IL-3 or IL-5, will bind to the β_{c} of the receptor. The binding motif of the receptor is then phosphorylated and preferably phosphorylates the ⁵⁸⁵Ser or equivalent residue. 14-3-3 can bind to the phosphorylated motif thereby positioning the 14-3-3 close to the receptor for further binding of cytoplasmic proteins involved in cell signaling (signaling molecules) for cellular activities such as proliferation, differentiation, cell survival, chemotaxis, motility, enhanced phagocytosis, bacterial killing, superoxide production and cytotoxicity.

In yet another aspect of the present invention there is provided a method of regulating cellular activities, said method including:

regulating the phosphorylation of a binding motif of a receptor as described above, a functional equivalent or analogue thereof;

subjecting the binding motif to a cytoplasmic protein to bind the cytoplasmic protein to the binding motif; and

activating a cell signaling pathway by interacting the bound cytoplasmic protein with a signaling molecule involved in the pathway.

Preferably, the cytoplasmic protein is 14-3-3 protein, or any cytoplasmic protein capable of binding a further signaling molecule which activates a cascade of events leading to cell signaling pathways and biological functions such as mitogenesis, proliferation, transformation, differentiation, cell survival, chemotaxis, motility, enhanced phagocytosis, bacterial killing, superoxide production and cytotoxicity. Preferably the cytoplasmic binding protein is 14-3-3.

There are many signaling molecules involved in cellular pathways leading to cellular activity. However, it is preferred in the present invention to provide a molecule that binds to a phospho-serine bound 14-3-3 molecule such that a pathway is coupled to the motif or equivalent unit in a receptor and brought into close proximity to downstream signaling proteins at, or near, the cell membrane. Cellular activities may include cell survival, proliferation, transformation, differentiation, mitogenesis, chemotaxis, motility, enhanced phagocytosis, bacterial killing, superoxide production and cytotoxicity.

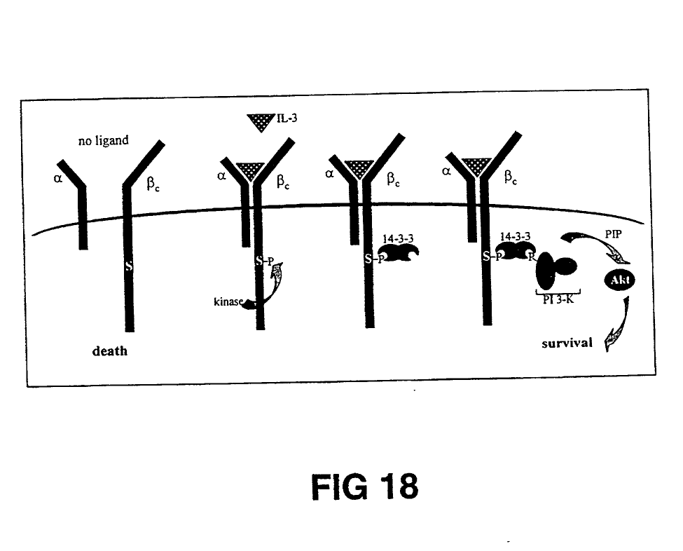

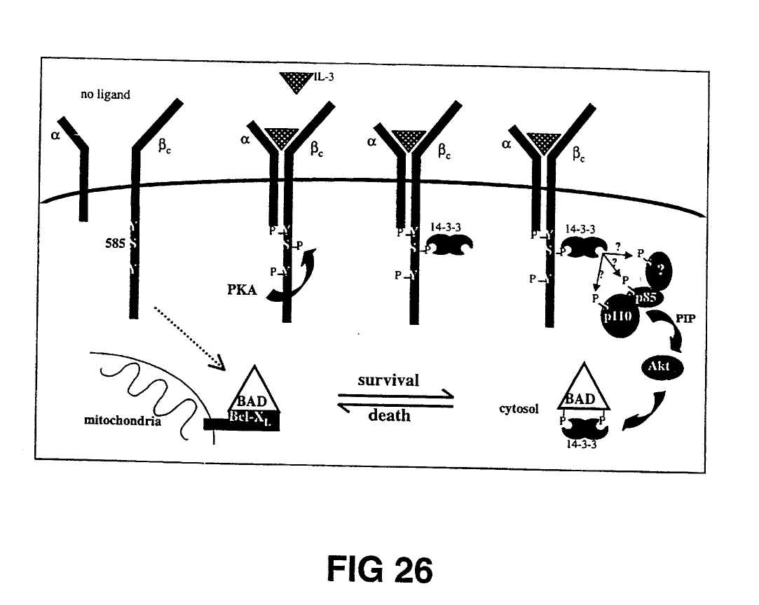

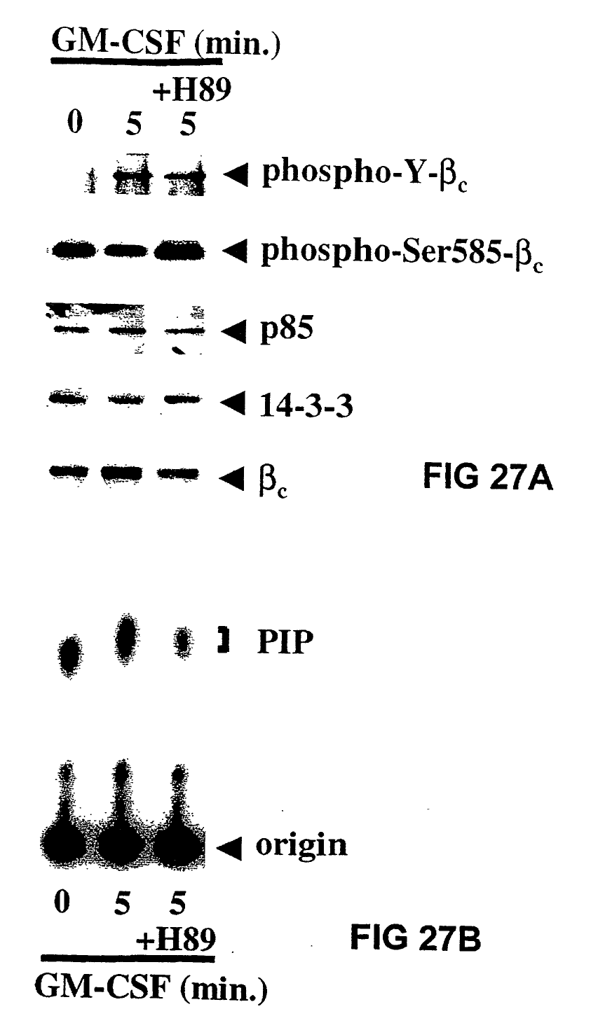

For regulating cell survival, it is preferred to activate the PI-3-kinase pathway using a PI-3 kinase bound to a phosphoserine bound 14-3-3. This may be shown in FIG. 18 or FIG. 26. FIG. 26 shows proposed model for the regulation of survival by IL-3. Binding of IL-3 to the IL-3 receptor, composed of a ligand specific α-chain (α) and a common β-chain (β_{c}), results in receptor oligomerization (only one αchain and one β-chain are shown for simplicity). Receptor oligomerization results in the activation of tyrosine kinases which results in tyrosine phosphorylation of β_{c} (P-Y) and the recruitment of SH2- and PTB-binding proteins (not shown). In addition, activation of PKA results in the phosphorylation of Ser585 of β_{c} (S-P). Ser585 phosphorylation allows the recruitment of 14-3-3, which in turn recruits PI 3-kinase (PI 3-K) either directly through the p110 or p85 subunits or indirectly through an additional adaptor molecule(s). These receptor proximal events identified in the current studies which result in the activation of PI 3-kinase are then likely to couple to a downstream pathway involving Akt and BAD. BAD phosphorylation results in 143-3 binding and sequestration of BAD in the cytoplasm and suppression of apoptosis (survival). In the absence of cytokine, BAD remains unphosphorylated and translocates to the mitochondria where the events leading to apoptosis are triggered (death).

Phosphorylation of the motif may be regulated by any means which results in inhibition or activation of the phosphorylation of the cytoplasmic protein binding motif particularly the Ser⁵⁸⁵ residue. Preferably, the phosphorylation is induced by a triggering molecule such as a cytokine selected from the group including GM20 CSF, IL-3 or IL-5. More preferably for cell survival, it is induced by IL-3. In this case, the β_{c} is phosphorylated. More preferably the binding motif comprising the amino acid sequence HSRSLP is phosphorylated. More preferably the second serine from the 5′ end is phosphorylated. In Pc, this correlates to ⁵⁸⁵Ser.

Regulation of cell survival may include enhancing or reducing cell survival or even causing cell death. This may be achieved by enhancing or inhibiting any of the steps described above. For instance enchancing phosphorylation of the binding motif may enhance survival. Alternatively, inhibiting phosphorylation may inhibit cell survival.

In another aspect of the present invention, there is provided a method of inhibiting cell survival, said method including inhibiting the binding of a cytoplasmic protein to a binding motif of a receptor as described above.

Preferably the cytoplasmic protein is 14-3-3.

Preferably the receptor is the GM-CSF/IL-5/IL-3 receptor and the triggering molecule is IL-3, although a phosphorylation event which phosphorylates ⁵⁸⁵Ser may also trigger the binding of 14-3-3 to the motif.

For cell survival or cell activation, it is preferred that the binding motif is as described above.

Antagonists that bind to the receptor motif in either the phosphorylated or unphosphorylated form may be useful to inhibit cell survival or activation. Preferably antagonists may be useful to inhibit cell survival or activation by preventing phosphorylation preferably by preventing serine phosphorylation of the β_{c} or equivalent thereby preventing the cytoplasmic protein binding to the binding motif. Alternatively, they may prevent the interaction of a signaling molecule binding to a phosphoserine bound 14-3-3 or equivalent. Prevention of phosphorylation of the β_{c} or binding motif as described above may be by inhibition of the specific kinases involved in the phosphorylation of the serine/threonine residue or it may include mutation of the binding motif to prevent the cytoplasmic protein such as 14-3-3 from binding and activating cell cycle pathways. Kinase inhibitors such as H89 which binds to PKA may be used. Preferably, cell permeable kinase inhibitors are used. Preferably the signaling molecule is a PI-3-kinase involved in the PI-3-kinase pathway which leads to cell survival or cell activation.

Antagonists may include antibodies, small peptides, small molecules, peptide mimetics or any type of molecule known to those skilled in the art that are directed to the cytoplasmic binding motif so as to prevent attachment of cytoplasmic proteins such as 14-3-3 to a phosphoserine residue or a signaling molecule. Antibodies may be generated in response to any of the binding motifs described above by methods known and available to the skilled addressee.

Hence, the antagonists as described may be useful as cancer therapeutics to prevent cell survival of cancer cells or cell activation such as myeloid cell activation and may be useful for preventing or treating leukaemia such as acute myeloid leukaemia (AML). Other uses of antagonists may be in prevention and treatment of inflammatory diseases.

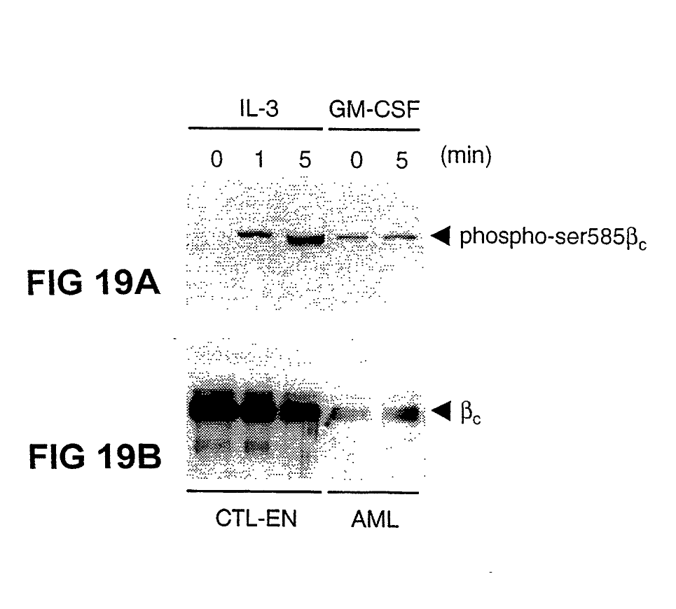

Applicants have also found that the ⁵⁸⁵Ser in β_{c} of acute myeloid leukaemia (AML) is constitutively phosphorylated (see FIG. 19). Hence by preventing phosphorylation of the ⁵⁸⁵Ser, further binding of 14-3-3 to the binding motif of β_{c} may be prevented thereby further preventing the binding of PI-3-kinase which interacts with the PI-3-kinase pathway to activate cell survival. This may be useful to prevent those functions related to cell activation, particularly myeloid cell activation. The functions may be selected from the group including chemotaxis, motility, enhanced phagocytosis, bacterial killing, superoxide production and cytoxicity. These functions may also contribute to inflammation including, but not limited to, asthma and rheumatoid arthritis.

Accordingly, there is provided a method of inhibiting cell activation, said method including inhibiting the binding of a cytoplasmic protein to a binding motif of a receptor as described above.

Accordingly this method of interaction may be a useful tool for a method of treating or preventing cell proliferative diseases such as AML or cancer.

Targeting may be by way of the use of antagonists as described above or by any means that prevents activation of cell cycles via the binding motif described in the present invention. Targeting may be by blocking or mutating the motif.

Techniques such as gene therapy may also be employed which targets or removes the binding motif.

In another aspect, there is also provided a method of treating a cytokine mediated condition said method comprising:

regulating the activation of phosphorylation of a binding motif of a receptor as described above, a functional equivalent or analogue thereof.

The cytokine mediated condition is a condition which requires a cytokine to bind to its receptor to induce a cellular activity. By regulating the activator, cellular action may be activated to increase the phosphorylation or to decrease phosphorylation. Preferably, the cytokine mediated condition is a GMCSF/IL-5 mediated condition and the binding motif includes the amino acid sequence ⁵⁸²HSR SLP⁵⁸⁷ on the β_{c}.

The present invention will now be more fully described with reference to the following examples. It should be understood, however, that the description following is illustrative only and should not be taken in any way as a restriction on the generality of the invention described above.

### EXAMPLE 1

### Binding of 14-3-3 to a Common Beta Chain (β_{c})

(a) Mutagenesis of Human β_{c} and Expression Plasmid Constructs.

Substitution mutations of two sequences within the cytoplasmic domain of the human β_{c} cDNA were constructed using oligonucleotide-directed mutagenesis (Altered-sites, Promega, Sydney, NSW, Australia) as described in Wood Cock, J. M. et al (1994) EMBO, 13, 5176. Both mutants were essentially poly-alanine substitutions. Mutagenesis oligonucleotides encoding non-alanine residues were included to facilitate restriction enzyme screening of mutant clones. The first motif was ⁵⁸²HSRSLP⁵⁸⁷ mutated to 582EFAAAA⁵⁸⁷, and the second was ⁸²⁰RSKPSSP⁸²⁶ mutated to ⁸²⁰EFAAAAA⁸²⁶. The point mutant S585A was also constructed, however this mutant created a cryptic proteolytic site in β_{c} and was not able to be used. The mutations were confirmed by nucleotide sequencing and the mutant β_{c} cDNAs suloned into the eukaryotic expression vector pcDNA1 (Invitrogen, San Diego, Calif.). The β_{c} deletion mutant cDNAs were a kind gift of Dr A. Miyajima.

(b) GM-CSF and IL-3.

Recombinant human IL-3 and GM-CSF were produced in E. coli essentially as described in Barry, S. C. et al (1994) J. Biol chem, 269, 8488 and Hercus, T. R. et al (1994) Proc. Nati. Acad. Sci., 91, 5838. Cytokine purity and quantitation was determined by HPLC analysis and Coomasie staining of SDS-PAGE separated proteins. The activity of the cytokines based on the ED₅₀ values in a TF-1 proliferation assay (Kitamura, T. et al (1989) J. Cell Physiol, 140, 323) was 0.03 ng/ml for GM-CSF and 0.1 ng/ml for IL-3.

(c) Antibodies.

The monoclonal antibodies 8E4 and 1C1 directed against the β_{c} were generated as previously described in Stomski, F. C. et al (1998) J. Biol. Chem., 273, 1192. The anti-phospho-⁵⁸⁵Serβ_{c} antibody was raised by immunising New Zealand white rabbits with the phosphorylated CLGPPHSRSLPDILG peptide conjugated to keyhole limpet hemocyanin (Sigma). The anti-peptide antibody was firstly affinity purified with the immunising peptide conjugated to sepharose and then absorbed with the nonphosphorylated CLGPPHSRSLPDILG peptide conjugated to sepharose. The specificity of the anti-phospho-⁵⁸⁵Serβ_{c} antibody was verified by dot immunoblots against the CLGPPHSRSLPDILG peptide either non phosphorylated or serine phosphorylated form and a scrambled peptide LPLSGPDSHIRGPL. The corresponding phosphorylated serine in each peptide is underlined. Peptides were synthesised by Chiron Mimotopes, Melbourne, Australia. The anti-14-3-3z antibody was kindly provided by Dr A. Aitken.

(d) Cell Culture and DNA Transfection.

The HEK293T cell line was maintained in RPMI-1640 supplemented with 10% v/v fetal calf serum (FCS). On the day before transfection, 1.4×10⁶ cells were plated into 6 cm tissue culture dishes to adhere overnight. Four hours after a medium change, 6 mg of wild type or mutated β_{c} cDNA was added to cells in the form of a calcium phosphate precipitate (Graham, F. L. et al (1973) Virology, 52, 456), and the cells were placed in an incubator for 4-6 h to permit the uptake of the DNA-calcium phosphate precipitate. The cells were then washed, replated and placed in the incubator for 48 h prior to cytokine treatment. M1 cell line expressing GM-CSF receptor alpha chain and β_{c} wild type was maintained in RPMI-1640 supplemented with 10% v/v FCS. The M1 cell line was kindly provided by Dr N. Nicola.

(e) Surface Marker Analysis by Flow Cytometry.

Expression of receptors on transfected cells was verified by flow cytometry. Briefly, cells were incubated with the anti-β_{c} MAb (1C1) (Stomski, F. C. et al (1996) Mol. Cell. Biol, 16, 3035) or anti-GM-CSFRa MAb (4H1) (Stomski, F. C. et al (1998) J. Biol. Chem. 273, 1192) for 20 minutes on ice, washed, and subsequently incubated with fluorescein isothiocyanate (FITC)-conjugated anti-mouse IgG antibody (Silenus Laboratories, Hawthorn, Victoria, Australia) for 20 minutes on ice. Cells were then washed and resuspended in FACS FIX and analysed using a Profile II (Coulter Electronics).

(f) Immunoprecipitations.

Cells were lysed in lysis buffer [150 mM NaCl, 10 mM Tris-HCl (pH 7.4), 1% Digitonin with protease inhibitors (10 mg/ml leupeptin, 2 mM phenylmethlysulfonyl fluoride, 10 mg/ml aprotinin) and 2 mM sodium vanadate] for 30 mins at 4° C. followed by centrifugation of the lysate for 15 mins at 12,000 g at 4° C. Following a 1 hour preclearance with Protein-A-sepharose (Pierce, Rockford, Ill.) at 4° C., the supernatant was incubated for 2 hours with 5 mg/ml antibody. Protein-immunoglobulin complexes were captured by incubation for 1 hour with Protein-A-sepharose followed by 6 subsequent washes in lysis buffer. Samples were boiled for 5 mins in SDS sample load buffer in the presence or absence of 2-mercaptoethanol before separating immunoprecipitated proteins by SDS-PAGE.

(g) Precipitations.

Cells were lysed in lysis buffer for 30 mins at 4° C. followed by centrifugation of the lysate for 15 mins at 12,000 g 4° C. Following a 1 hour preclearance with GST-sepharose at 4° C., the supernatant was incubated for 2 hours with GST-14-3-3-sepharose followed by 3 subsequent washes in lysis buffer. Samples were boiled for 5 mins in SDS sample load buffer in the presence of 2-mercaptoethanol before separating precipitated proteins by SDS-PAGE.

(h) Competition of Precipitations and Immunoprecipitations by Peptides.

Cell lysates were precipitated or immunoprecipitated in the presence of various concentrations of the following peptides: β_{c} peptide sequence CLGPPHSRSLPDILG either non phosphorylated or serine phosphorylated and a scrambled peptide CLPLSGPDSHIRGPL. Raf1 peptides corresponding to the sequence CLSQRQRSTSTPNVHM were also used and were either non phosphorylated or serine phosphorylated. The corresponding phosphorylated serine in each peptide is underlined. Peptides were synthesised by Chiron Mimotopes, Melbourne, Australia. The presence of β_{c} in either the immunoprecipitation or precipitation experiment was determined by Western blotting with anti-β_{c} antibody (MAb 1C1).

(i) SDS-Polyacrylamide Gel Electrophoresis, Immunoblot and ECL

Immunoprecipitated proteins separated by SDS-PAGE were transferred to nitrocellulose membrane by electroblotting. Dot blots of peptides were performed by spotting either 10 or 100 ng of each peptide onto nitrocellulose membranes. Routinely, nitrocellulose membranes were blocked in a solution of PBS/0.05% (v/v) Tween 20 (PBT) containing 1% (w/v) blocking reagent 1096 176 (Boehringer Mannheim) and probed with anti-β_{c} (1C1), anti-14-3-3z³³ or anti-phospho⁸⁵⁵Serβ_{c} followed by either anti-mouse or rabbit peroxidase conjugated antibodies. Immunoreactive proteins were detected by chemiluminescence using the ECL kit (Amersham, Little Chalfont, U.K.) following the manufacturer's instructions.

(j) Binding of the ¹²⁵I-Labeled 14-3-3z to Synthetic Peptides.

The recombinant 14-3-3z protein was ¹²⁵¹-labeled using IODOBEADS (Pierce). The synthetic peptides were solubilised in distilled water and then diluted to 50 mg/ml in 0.1 M NaHCO₃, pH 9.2. The peptides were coated onto microtiter wells (Immunolon II Removawells, Dynatech Laboratories, Chantilly, Va.) by incubation at 22° C. for 6 h and then 4° C. overnight. The peptide coated microtiter wells were blocked at 22° C. with 5% bovine serum albumin for 2 h and then with ¹²⁵I-labeled 14-3-3z protein for 2 h. After three washes, microtiter well-bound radioactivity were estimated in a g-counter.

The results of these experiments show that that firstly, β_{c} interacts with 14-3-3, and secondly, that the region of interaction lies between amino acids 544 and 626.

### EXAMPLE 2

### Binding of 14-3-3 to ⁵⁸²HSRSLP⁵⁸⁷ Motif of β_{c}

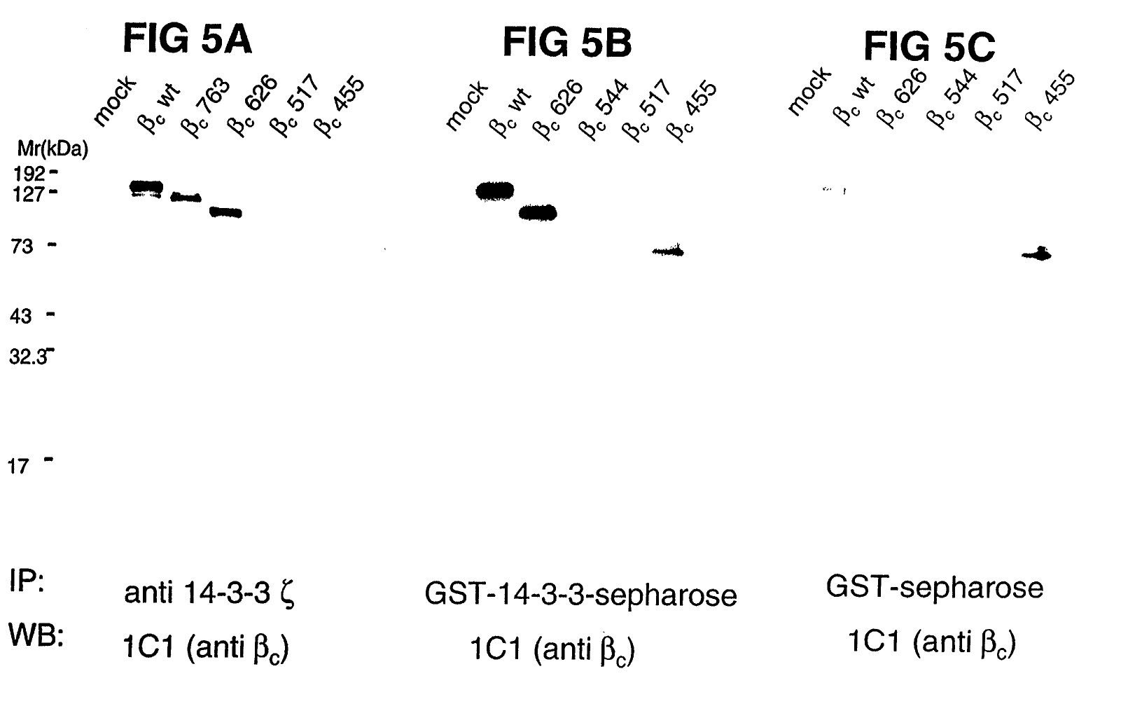

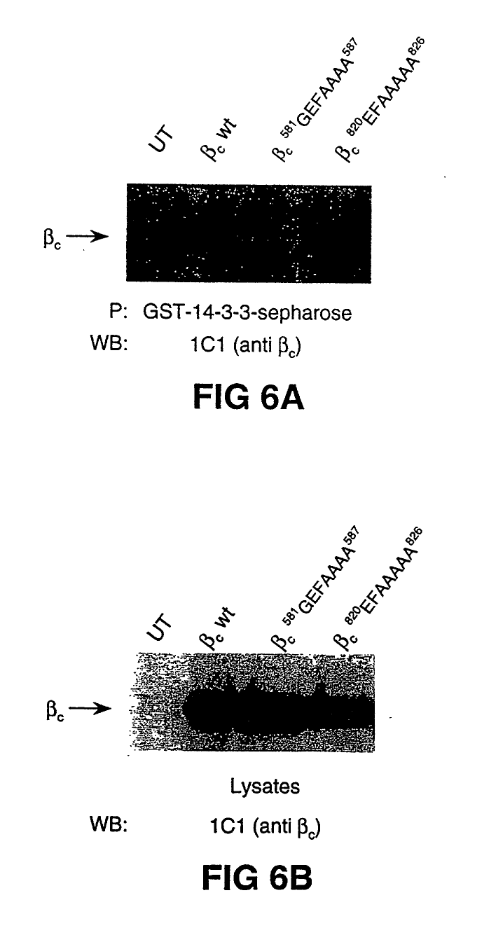

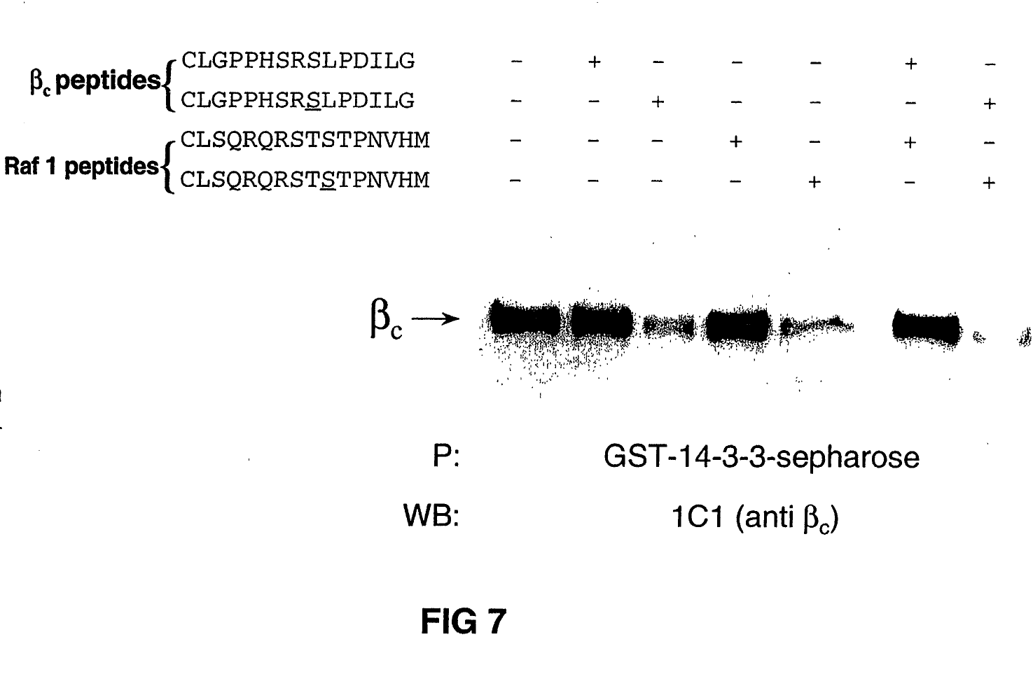

We then examined whether 14-3-3 interacted with β_{c} via the ⁵⁸²HSRSLP⁵⁸⁷ motif which lies within the 544-626 region identified in FIG. 5. A substitution mutant (β_{c}-⁵⁸²HSRSLP⁵⁸⁷→EFAAAA) and a point mutant (β_{c}-585S→A) in the putative 14-3-3 binding site were constructed as well as a control mutant (β_{c} ⁸²⁰RSKPSSP⁸²⁶→EFAAAAA). These mutants were expressed in HEK 293T cells and examined for their ability to interact with GST-14-3-3 in pull-down experiments. While wild type β_{c} and the control mutant β_{c} interacted with GST -14-3-3, no detectable interaction was observed for the β_{c} ⁵⁸²HSRSLP⁵⁸⁷→EFAAAA mutant (FIG. 6). These results indicate that 14-3-3 associates with β_{c} via the ⁵⁸²HSRSLP⁵⁸⁵ sequence.

### EXAMPLE 3

### Role of ⁵⁸⁵ SER in β_{c} Interaction with 14-3-3

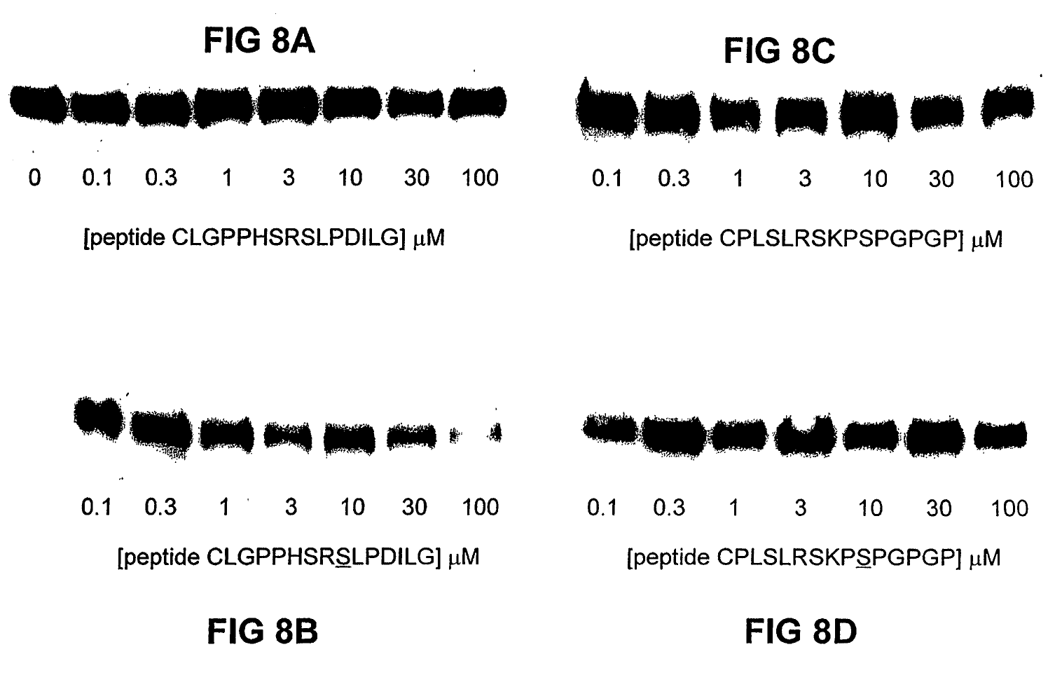

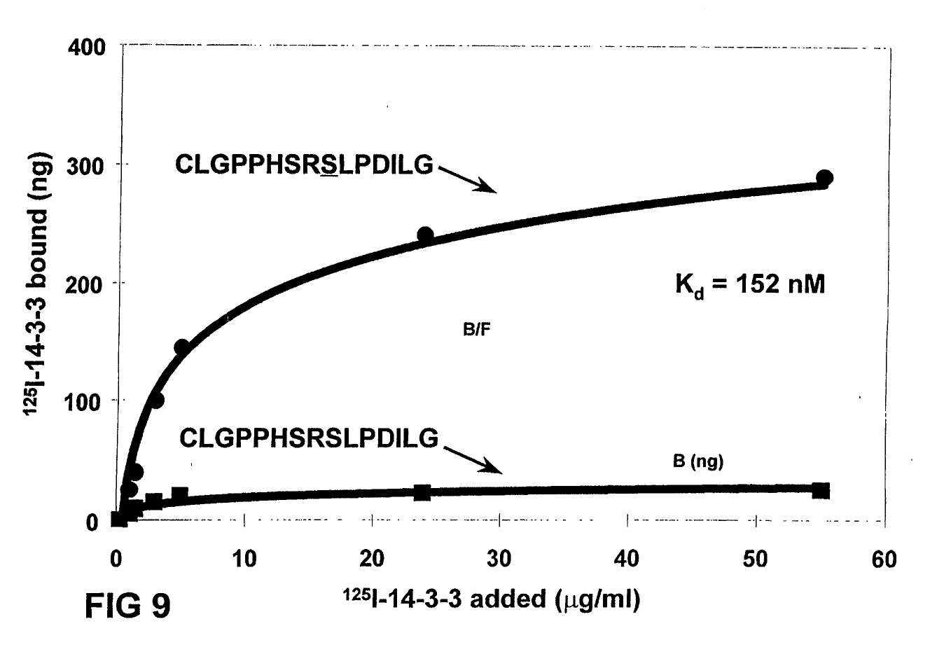

14-3-3 is known to be a phospho-serine binding protein which interacts with the RSXSXP motif, where S is phosphorylated. We tested whether ⁵⁸⁵Ser phosphorylation within the ⁵⁸²HSRSLP⁵⁸⁷ motif would be required for 14-3-3 association. We synthesised a β_{c} peptide containing a non-phosphorylated ⁵⁸⁵Ser (C⁵⁷⁸LGPPHSRSLPDILG⁵⁹¹) and a β_{c} peptide containing a phosphorylated ⁵⁸⁵Ser and examined their ability to inhibit β_{c} interaction with GST-14-3-3 in a pull-down experiment. While the peptide containing phosphorylated ⁵⁸⁵Ser inhibited β_{c} association with GST-14-3-3, no inhibition of association was observed for the peptide containing the non-phosphorylated ⁵⁸⁵Ser (FIG. 4). As a comparison, nonphosphorylated and phosphorylated peptides corresponding to the 14-3-3 binding site in Raf-1 were also tested. We found that the serine phosphorylated Raf-1 peptide was also able to inhibit β_{c} association with 14-3-3 while the nonphosphorylated peptide did not (FIG. 4). Furthermore, the ability of the β_{c} phosphorylated peptide to inhibit the association of β_{c} with 14-3-3 was dose-dependent and specific as another phosphorylated peptide with sequence corresponding to a different region of β_{c} failed to inhibit this association (FIG. 8). Direct binding experiments and Scatchard analysis demonstrated that the phosphorylated peptide (C⁵⁷⁸LGPPHSRSLPDILG⁵⁹¹) bound to 14-3-3 with an affinity of approximately 150 nM (FIG. 9). These experiments show that the ⁵⁸²HSRSLP⁵⁸⁷ sequence in β_{c} directly binds to 14-3-3 and that this binding is dependent on ⁵⁸⁵Ser being phosphorylated.

### EXAMPLE 4

### In Vivo Regulation of ⁵⁸⁵SER Phosphorylation

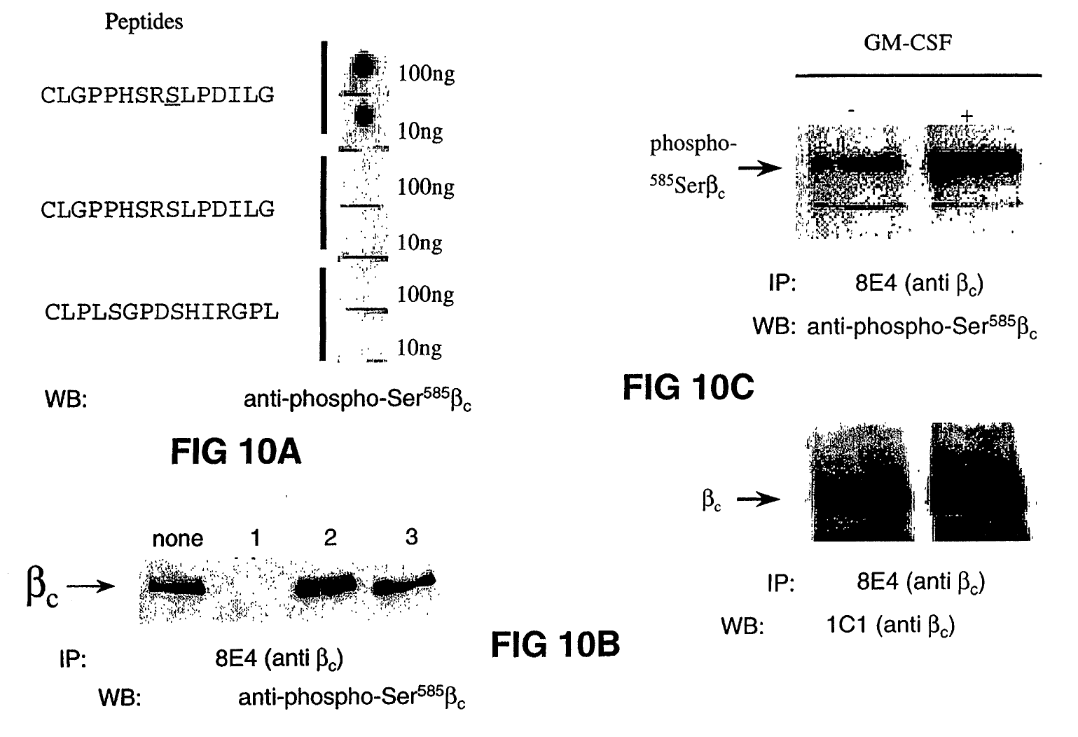

Having identified the requirement for β_{c} ⁵⁸⁵Ser to be phosphorylated to allow 14-3-3 binding, we then examined whether ⁵⁸⁵Ser was phosphorylated in vivo and whether its phosphorylation was regulated by GM-CSF. These possibilities were initially addressed using ³²P-orthophosphate labelled HEK 293T cells transfected with the GM-CSF receptor (α and β_{c} subunits). These cells were stimulated with GM-CSF, total β_{c} was immunoprecipitated and examined for ³²P-labelling. This is most likely due to the presence of sixty serine and threonine residues in the intracellular region of β_{c}, some of which may be constitutively phosphorylated. In order to directly address the phosphorylation status of β_{c} ⁵⁸⁵Ser in vivo, we raised a rabbit antiserum against a peptide containing the 14-3-3 binding site identified in β_{c}: C⁵⁷⁸LGPPHSRSLPDILG⁵⁹¹. This antibody preparation, termed anti-phospho-⁵⁸⁵Ser specifically recognised the CLGPPHSRSLPDILG peptide containing a phosphorylated ⁵⁸⁵Ser but not a peptide containing a non-phosphorylated ⁵⁸⁵Ser or a peptide containing a scrambled 14-3-3 binding site (FIG. 10). The specificity of the anti-phospho-⁵⁸⁵Ser antibody was further confirmed by Western blotting of immunoprecipitated β_{c} from GM-CSF receptor-transfected HEK 293T cells. In these experiments, the phosphorylated CLPPHSRSLPDILG peptide was able to inhibit anti-phospho-⁵⁸⁵Ser recognition of β_{c}, while the non-phosphorylated and scrambled peptides did not (FIG. 10b). In addition, pretreatment of β_{c} immunoprecipitates with calf intestinal phosphatase prior to Western blot analysis completely abolished the anti-phospho-⁵⁸⁵Ser signal and immunoprecipitation of either the wild type β_{c} or the ⁵⁸²HSRSLP⁵⁸⁷→EFAAAA mutant from HEK293T transfected cells followed by western blot analysis with the anti-phospho-⁵⁸⁵Ser antibody demonstrated that this antibody specifically recognised the wild type but not the mutant receptor (data not shown).

Using these anti-phospho-⁵⁸⁵Ser specific antibodies we then examined the regulation of β_{c} ⁵⁸⁵Ser phosphorylation following GM-CSF stimulation of MI myeloid leukaemic cells. M1 myeloid leukaemic cells were stimulated with 2 ng/ml GM-CSF, β_{c} was immunoprecipitated, and immunoprecipitates probed with the anti-phospho-⁵⁸⁵Ser antibody. As shown in FIG. 10, GM-CSF stimulation strongly upregulated ⁵⁸⁵Ser phosphorylation of β_{c}.

### EXAMPLE 5

### IL-3 Induces Ser585 Phosphorylation of β_{c} and Recruitment of 14-3-3.

We have described the biochemical interaction of the β_{c} of the GM-CSF, IL-3 and IL-5 receptors with the 14-3-3 family of phospho-serine binding proteins. These studies identified a motif, ⁵⁸²HSRSLP⁵⁸⁷, in the cytoplasmic domain of β_{c} that binds 14-3-3 when Ser585 is phosphorylated. To address the functional significance of 14-3-3 binding to β_{c} we generated cell lines expressing either wild type or mutant IL-3 receptors lacking the 14-3-3 binding site. CTL-EN cells were tranduced with viral constructs expressing IL-3Ra and either wtβ_{c}, a substitution mutant that encompasses the 14-3-3 binding site (β_{c}HSRSLP→EFAAAA) or a point mutant in which Ser585 of the 14-3-3 binding motif was substituted for alanine (β_{c}S585A). The β_{c}S585A point was not able to be used in these studies as it appeared to introduce a cryptic proteolytic cleavage site. Western blot analysis and flow cytometry indicated that this mutant was proteolysed and failed to be expressed on the cell surface (Stomski et al., 1999) Blood, 94, 1933-1942). Similar expression levels of wtβ_{c} and β_{c}HSRSLP→EFAAAA were observed in the CTL-EN cells by both flow cytometry (data not shown) and Western blot analysis (see below). Similar expression levels of IL-3Ra were also observed for each cell line.

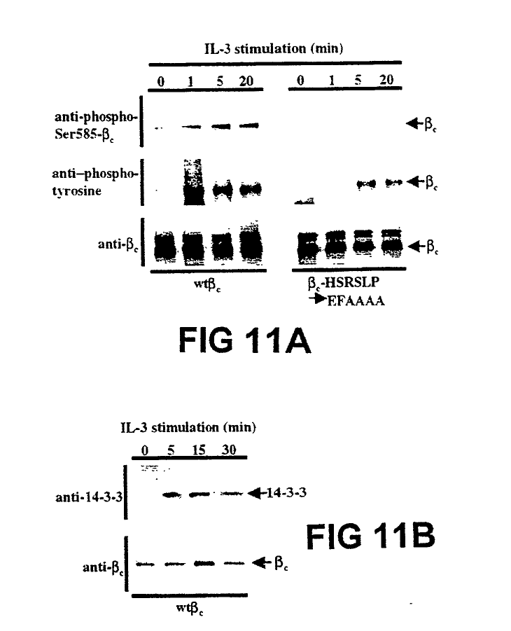

The regulation of β_{c}Ser585 phosphorylation in response to IL-3 stimulation was then examined in these cell lines using anti-phospho-Ser585β_{c} antibodies. These antibodies have been shown to specifically recognize the phosphorylated 14-3-3 binding motif HSRSLP (where S is phosphorylated) but not the non-phosphorylated motif. CTL-EN cells expressing wtβ_{c} or β_{c}HSRSLP→EFAAAA were starved for 4 hours in 0.1% FCS and then stimulated with 50 ng/ml of IL-3. The β_{c} was immunoprecipitated and then subjected to immunoblot analysis using the anti-phospho-Ser585β_{c} antibodies. Ser585 phosphorylation was upregulated in response to IL-3 stimulation in CTL-EN cells expressing wt while no signal was detected for the β_{c}HSRSLP→EFAAAA mutant control (FIG. 11a). Tyrosine phosphorylation of β_{c} was detected for both the wtβ_{c} and the β_{c}HSRSLP→EFAAAA mutant (FIG. 11a). Co-immunoprecipitation experiments showed that increased β_{c}Ser585 phosphorylation in response to IL-3 stimulation also resulted in increased 14-3-3 binding to β_{c} (FIG. 11b). No association of 14-3-3 with the β_{c}HSRSLP→EFAAAA mutant control in response to IL-3.

### EXAMPLE 6

### cAMP-Dependent Protein Kinase (PKA) Phosphorylates Ser585 of

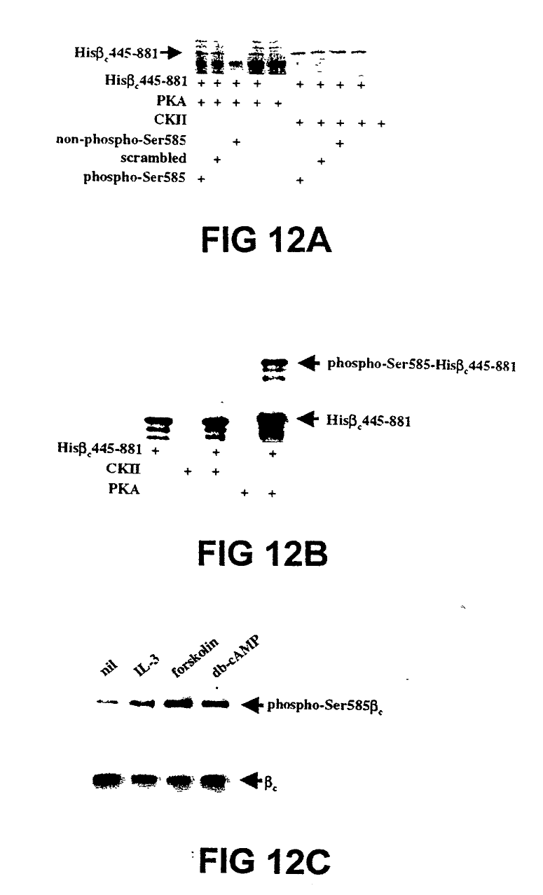

In an effort to identify the kinase involved in the phosphorylation of Ser585, we firstly examined the ability of several kinases to phosphorylate the purified recombinant intracellular portion of β_{c} (hisβ_{c}445-881) in vitro. Ser585 of β_{c} lies within a SXXD motif which is known to be a CKII consensus phosphorylation site. Ser585 also lies within a RS motif which is a possible, but poor, PKA phosphorylation site. In the in vitro kinase assay shown in FIG. 12a, ³²P-labelling of hisβ_{c}445-881 was observed for both PKA and CKII. Additional bands for the PKA kinase reactions were evident due to the autocatalytic activity of PKA. Interestingly, the presence of a non-phospho-HSRSLP peptide reduced the phosphorylation of hisβ_{c}445-881 by PKA whereas a phospho-HSRSLP or a scrambled peptide did not (FIG. 12a). In this experiment, the non-phospho-HSRSLP peptide is likely to act as a competitive substrate for phosphorylation of Ser585 of hisβ_{c}445-881 by PKA. The phospho-HSRSLP and scrambled peptides would not act as competitive substrates and therefore did not result in decreased phosphorylation of hisβ_{c}445-881. On the other hand, the phosphorylation of hisβ_{c}445-881 by CKII was not inhibited by any of the peptides suggesting that CKII cannot phosphorylate Ser585 of hisβ_{c}445-881 (FIG. 12a).

To further examine the possibility that PKA could phosphorylate Ser585 of β_{c}, we performed in vitro kinase assays using cold ATP and examined the phosphorylation of Ser585 by immunoblot analysis using the anti-phospho-Ser585β_{c} antibodies. CKII was not able to detectably phosphorylate Ser585 of hisβ_{c}445-881 whereas PKA resulted in strong phosphorylation of Ser585 as detected by the anti-phospho-Ser585 antibody (FIG. 12b). The results shown in FIGS. 12a and 12b support the proposal that PKA can phosphorylate Ser585 of β_{c} in vitro. We then examined the ability of PKA to phosphorylate Ser585 of β_{c} in vivo. CTL-EN cells expressing wtβ_{c} were starved for 12 hours in DMEM containing 0.5% FCS and then either left unstimulated (nil), stimulated with 50 ng/ml IL-3, 50 mM forskolin or 100 m dibutyryl cAMP for 10 minutes. Cells were lysed, β_{c} was immunoprecipitated and subjected to immunoblot analysis with the anti-phospho-Ser585 antibody. IL-3 stimulation resulted in increased Ser585 phosphorylation (FIG. 12c, IL-3). In addition, agents that elevate intracellular levels of cAMP and activate PKA were also found to result in increased Ser585 phosphorylation of β_{c}. Together, the results shown in FIG. 12 would suggest that PKA is likely to phosphorylate Ser585 of β_{c} in response to IL-3 stimulation.

### EXAMPLE 7

### 14-3-3 Recruitment to β_{c} in Response to IL-3 is Important for the Activation of the PI 3-Kinase Pathway.

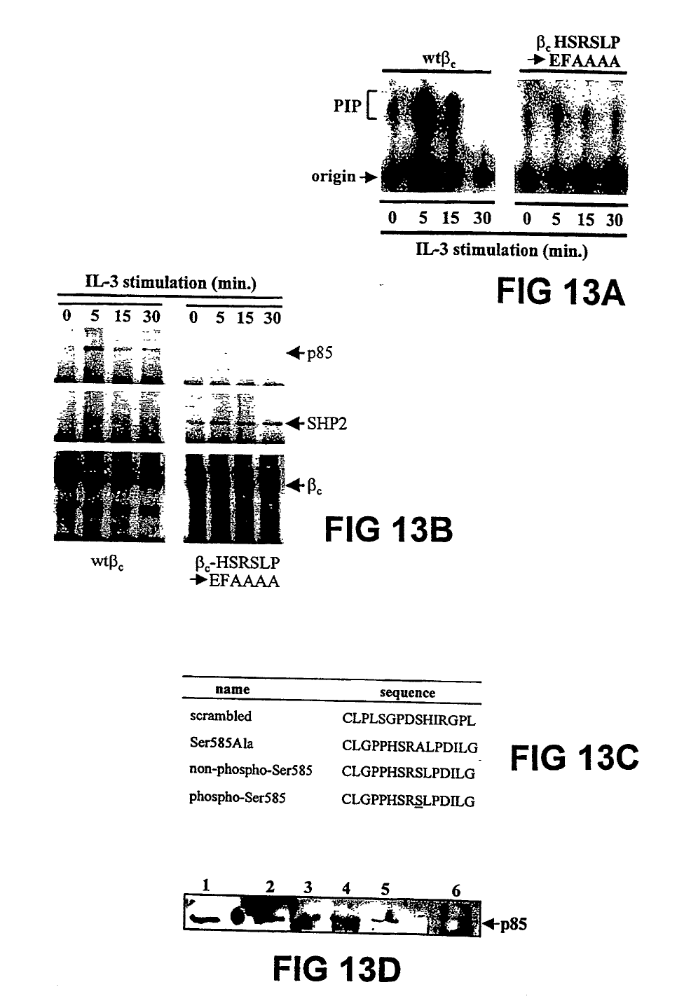

The regulation of Ser585 phosphorylation and the recruitment of the phosphoserine adaptor protein 14-3-3 to β_{c} raised the possibility that these events were important in regulating IL-3 signalling. GM-CSF and IL-3 are known to activate at least three pathways; the JAK/STAT pathway, the ras/MAP kinase pathway and the PI 3-kinase pathway (Guthridge et al., 1998 skin cells 16, 301-313). These pathways are not necessarily mutually exclusive and may have substantial overlap. The PI 3-kinase pathway has been implicated as having an important role in regulating cellular survival in a number of systems (Marte and Downward, (1997); Trends Biochem Sci 22, 355-358 cell, 88, 435-437) Franke et al., 1997) and, significantly, has been shown to bind 14-3-3 (Bonnefoy-Berard et al., (1995); Lui et al., 1996). We therefore examined the possibility that Ser585 of β_{c} and 14-3-3 maybe important in the regulation of the PI 3-kinase pathway. CTL-EN cells expressing wtβ_{c}, or β_{c}HSRSLP→EFAAAA were starved and stimulated with IL-3, tyrosine phosphorylated proteins were immunoprecipitated and the PI 3-kinase activity of the immunoprecipitates was determined in an in vitro kinase assay using phosphatidyl inositol and g³²P-ATP as substrates. While IL-3 stimulation of cells expressing wtβ_{c} resulted in the rapid and transient activation of PI 3-kinase activity, no PI 3-kinase activity was observed in response to IL-3 stimulation for the β_{c}HSRSLP→EFAAAA mutant indicating that 14-3-3 binding to is necessary for PI 3-kinase activation (FIG. 13a).

Although β_{c} does not contain a classical YXXM consensus for the direct binding of PI 3-kinase, our results raise the possibility of an alternative mechanism whereby 14-3-3 could act as an adaptor to recruit PI 3-kinase to β_{c} via phospho-Ser585. To examine this possibility we performed co-immunoprecipitation experiments examining the association of β_{c} with the p85 regulatory subunit of PI 3-kinase. CTL-EN cells expressing wtβ_{c} or β_{c}HSRSLP→EFAAAA were stimulated with IL-3 for up to 30 minutes, β_{c} was immunoprecipitated and the immunoprecipitates were examined for associated p85 subunit by immunoblot analysis. IL-3 stimulation resulted in the association of p85 with wtβ_{c} which was maximal at 5 minutes and was decreased by 30 minutes (FIG. 13b). In contrast, the recruitment of p85 to the β_{c}HSRSLP→EFAAAA mutant in response to IL-3 was virtually abolished (FIG. 13b). We also examined the ability of the mutant β_{c} to recruit the protein tyrosine phosphatase, SHP2. Previous studies have shown that Tyr612, which lies adjacent to the 14-3-3 binding site, is a likely binding site for SHP2 (Sakamaki K et al., 1992; Bone et al., 1997). Both the wtβ_{c} and the β_{c}HSRSLP→EFAAAA mutant receptors were able to recruit SHP2 in response to IL-3 indicating that the 14-3-3 binding mutant did not induce structural alterations that prevented the recruitment of other signalling molecules. These results suggest that 14-3-3 interaction with β_{c} is necessary for the recruitment of PI 3-kinase.

The interaction of β_{c} with PI 3-kinase was further addressed by examining the ability of the p85 subunit of PI 3-kinase to interact with purified his-tagged intracellular-portion of β_{c} (hisβ_{c}445-881) via the phosphorylated HSRSLP motif. hisβ_{c}445-881 expressed in bacteria is not phosphorylated on Ser585, however as shown in FIGS. 12a and 12b, we have found that cAMP-dependent protein kinase (PKA) can efficiently phosphorylate Ser585 of hisβ_{c}445-881 in vitro. hisβ_{c}445-881 coupled to Sepharose and phosphorylated on Ser585 by PKA was incubated with COS-7 cell lysates and examined for its ability to pull-down p85 by immunoblot analysis. In addition, a number of peptides encompassing the 14-3-3 binding site in β_{c} (listed in FIG. 3c) were examined for their ability to competitively inhibit the interaction of hisβ_{c}445-881 with p85 (FIG. 12c). hisβ_{c}445-881 Sepharose was able to pull down p85 from the COS-7 cell extracts (FIG. 13d, lane 1) and the presence of scrambled (FIG. 13d, lane 2), Ser585Ala (FIG. 13d, lane 3), or non-phosphorylated-Ser585 (FIG. 13d, lane 4) control peptides had no apparent effect on this interaction. On the other hand, the interaction of p85 with hisβ_{c}445-881 was markedly reduced in the presence of a phospho-Ser585 peptide (FIG. 13d, lane 5). Because the recombinant hisβ_{c}445-881 is not tyrosine phosphorylated and that only a phospho-Ser585 peptide could compete for p85 binding to β_{c}, these results suggest that Ser585 phosphorylation of β_{c} is responsible for the association of both 14-3-3 and p85.

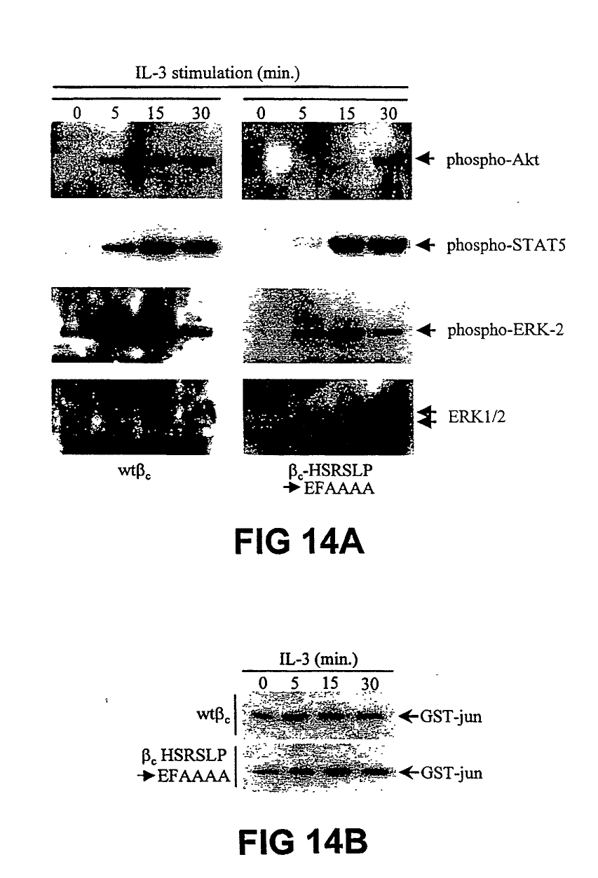

To further examine if 14-3-3 binding to β_{c} was involved in regulating other signalling pathways besides the PI 3-kinase pathway, we investigated a number of additional signalling molecules known to be activated in response to IL-3. CTL-EN cells expressing either wtβ_{c} or the β_{c}HSRSLP→EFAAAA receptors were starved and then stimulated with IL-3 and the activation of Akt (or protein kinase B), the signal transducer and activator of transription protein STAT5, and the extracellular-regulated kinases ERK1 and ERK 2 (or MAP kinase) was examined in whole cell lysates by Western blot using phospho-specific antibodies. Akt is a known downstream target of PI 3-kinase whereas STAT5 and ERK are thought to be regulated by pathways distinct from the PI 3-kinase pathway. CTL-EN cells expressing wtβ_{c} or the β_{c}HSRSLP→EFAAAA mutant induced STAT5 and ERK phosphorylation in response to IL-3 (FIG. 14a). While CTL-EN cells expressing wtβ_{c} demonstrated phosphorylation of Akt in response to IL-3, the phosphorylation of Akt was almost completely abolished in cells expressing the β_{c}HSRSLP→EFAAAA mutant (FIG. 14a). The activation of c-jun N-terminal kinase (JNK) was also examined in JNK immunoprecipitates using an in vitro kinase assay and GST-jun as a substrate. CTL-EN cells expressing either wtβ_{c} or β_{c}HSRSLP→EFAAAA were able to induce JNK activity in response to IL-3 (FIG. 14b). Together, these results show that phosphorylation of Ser585 of β_{c} and 14-3-3 association are important for the recruitment of PI 3-kinase to β_{c} as well as the activation of its downstream signalling partner Akt. Furthermore, Ser585 phosphorylation of β_{c} and 14-3-3 binding are important for the specific regulation of the PI 3-kinase pathway but not for the regulation of other known pathways emanating from β_{c} that utilize STAT5, ERK or JNK.

### EXAMPLE 8

### Association of 14-3-3 with β_{c} is Important for IL-3-Mediated Cell Survival but not Proliferation.

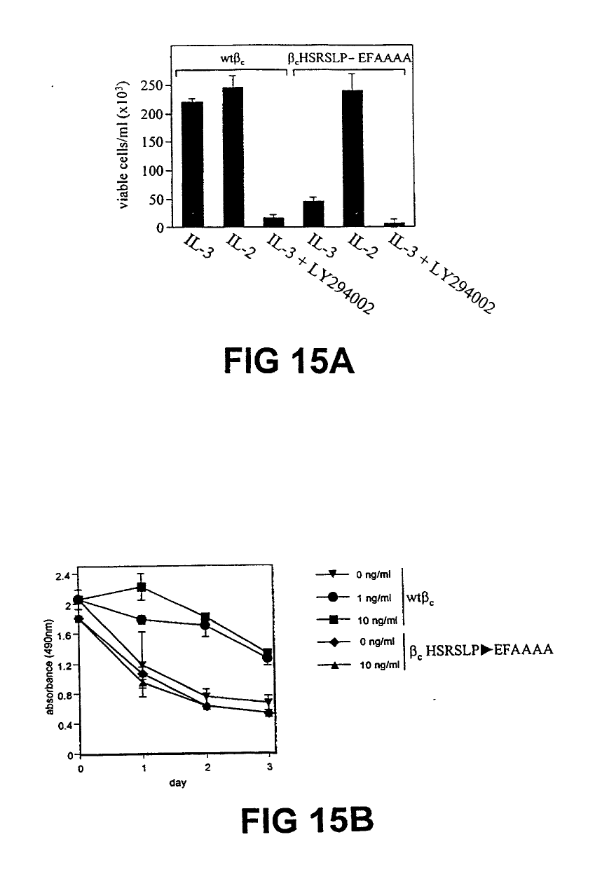

Given that 14-3-3 binding to Ser585 of β_{c} is required for PI 3-kinase activation in response to IL-3 and also the important role PI 3-kinase is thought to play in mediating survival signals, we then examined the ability of IL-3 to promote long-term survival in CTL-EN cells expressing the β_{c}HSRSLP→EFAAAA mutant receptor. Initial experiments performed in the presence of 10% FCS showed no defect in either the survival or growth of CTL-EN cells expressing the β_{c}HSRSLP→EFAAAA mutant receptor (data not shown). However, a clear defect in the survival of CTL-EN cells expressing the β_{c}HSRSLP→EFAAAA mutant was observed under low serum conditions. CTL-EN cells expressing the wt IL-3 receptor remain greater than 90% viable for up to 3-4 days under low serum conditions (0.1% FCS) in the presence of IL-3. To test the ability of IL-3 to promote cell survival, CTL-EN cells expressing either wtβ_{c} or β_{c}HSRSLP→EFAAAA were plated out at 2.5×10⁵ cells/ml in the presence of IL-3 or IL-2 in medium containing 0.1% FCS and viable cells were counted after 4 days. While CTL-EN cells expressing wtβ_{c} remained greater than 90% viable for up to 3-4 days in the presence of either IL-3 or IL-2, cells expressing the β_{c}HSRSLP→EFAAAA mutant showed a loss of viability in the presence of IL-3 with only 7% viable cells remaining after 4 days (FIG. 15a). Cells expressing the mutant receptor were able to maintain viability in the presence of IL-2. Importantly, CTL-EN cells expressing the wtβ_{c} lost viability in the presence of IL-3 and the PI 3-kinase inhibitor, LY294002, confirming that PI 3-kinase activity is essential for the survival these cells (FIG. 15a). As the defect in survival was apparent under low serum conditions (0.1% FCS) but not in the presence of 10% FCS, these results would suggest that there are factors in serum that can, at least in part, activate survival signals similar to those induced by IL-3. In fact, we have found that while serum is neither necessary for the promotion of CTL-EN cell survival (IL-3 in serum-free medium can promote survival of CTL-EN cells expressing the IL-3 receptor) nor sufficient in promoting survival (10% FCS fails to promote survival of CTL-EN cells expressing the IL-3 receptor) it can clearly augment the ability of IL-3 to stimulate cell survival (unpublished observations and these studies).

Loss of cell viability in CTL-EN cells expressing the β_{c}HSRSLP→EFAAAA in response to IL-3 was also reflected in loss of cellular metabolic activity as determined by the MTS reduction assay. CTL-EN cells expressing wtβ_{c} maintain appreciable levels of metabolic activity in the presence of IL-3 and 0.1% FCS for up to 3 days but lost metabolic activity in the absence of hIL-3 (FIG. 15b). CTL-EN cells expressing the β_{c}HSRSLP→EFAAAA mutant receptor lost metabolic activity in the presence of IL-3 (FIG. 15b). These data further suggest that the association of 14-3-3 with β_{c} in response to IL-3 is important for maintaining cellular viability.

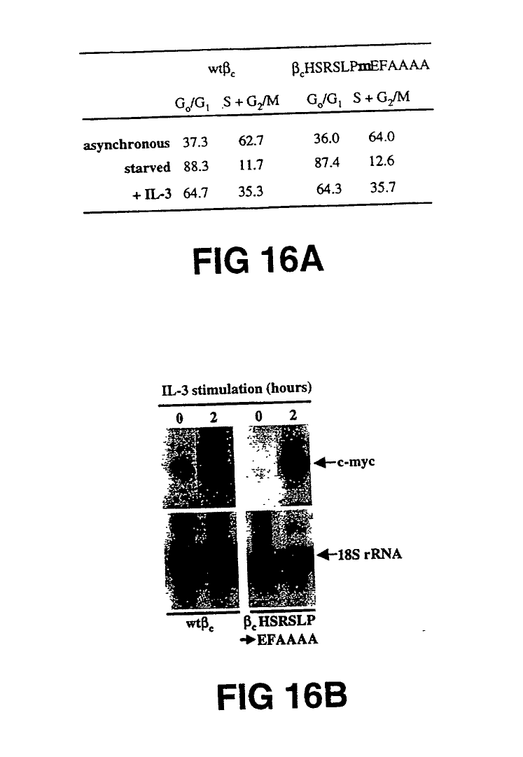

To determine if the signalling defect of the β_{c}HSRSLP→EFAAAA receptor was specifically related to cell survival or if cell proliferation was also impaired, cell cycle progression in response to IL-3 was examined. CTL-EN cells were starved in medium containing 0.1% FCS and no IL-3 for 24 hours and then stimulated with IL-3 for up to 24 hours. The cells were then fixed in ethanol and the cell cycle distribution was examined by prodidium iodide staining and flow cytometry. Under these starvation conditions, nearly 90% of CTL-EN cells accumulate in the G₀/G₁ phase of the cell cycle (FIG. 16a). CTL-EN cells expressing either wtβ_{c} or the β_{c}HSRSLP→EFAAAA mutant and synchronized in G₀/G₁ were both able to re-enter the cell cycle in response to IL-3 indicating that Ser585 phosphorylation, and 14-3-3 binding are not essential for signals that promote cell cycle progression (FIG. 16a). We also examined the regulation of c-myc mRNA expression in response to IL-3 by Northern blot analysis. The induction of c-myc is proposed to be an important pre-requisite for cell proliferation. No impairment of c-myc mRNA induction was observed in CTL-EN cells expressing β_{c}HSRSLP→EFAAAA when compared to control cells expressing wtβ_{c} (FIG. 16b).

### EXAMPLE 9

### Loss of 14-3-3 Binding to β_{c} Results in Cell Death by Apoptosis.

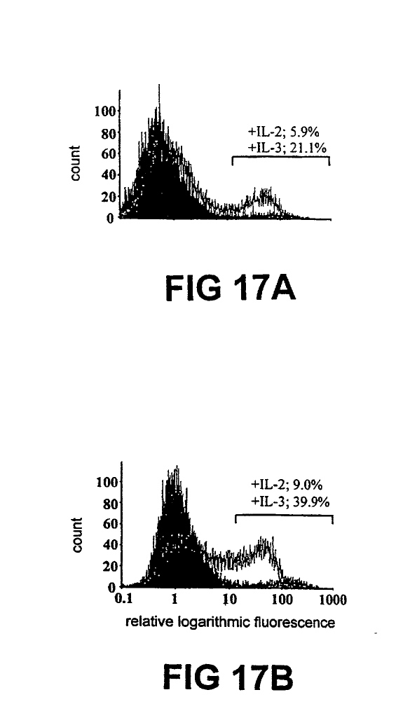

We then performed Annexin V binding studies to determine if the loss in cell viability in CTL-EN cells expressing the β_{c}HSRSLP→EFAAAA mutant was due to apoptosis. CTL-EN cells expressing wtβ_{c} or β_{c}HSRSLP→EFAAAA were washed and placed in medium containing 0.1% FCS and either IL-2 or IL-3 for 16 hours. The cells were then stained with annexin V and apoptotic cells were analysed by flow cytometry. CTL-EN cells expressing wtβ_{c} or β_{c}HSRSLP→EFAAAA showed negligible annexin V staining in the presence of IL-2 (FIG. 17). However, in the presence of IL-3, CTL-EN cells expressing the β_{c}HSRSLP→EFAAAA mutant showed increased annexin V staining compared to cells expressing the wtβ_{c} indicating that the defect in cell survival is due to increased apoptosis (FIG. 17).

### EXAMPLE 10

### IL-3 Induces Ser585 Phosphorylation of β_{c} and Recruitment of 14-3-3.

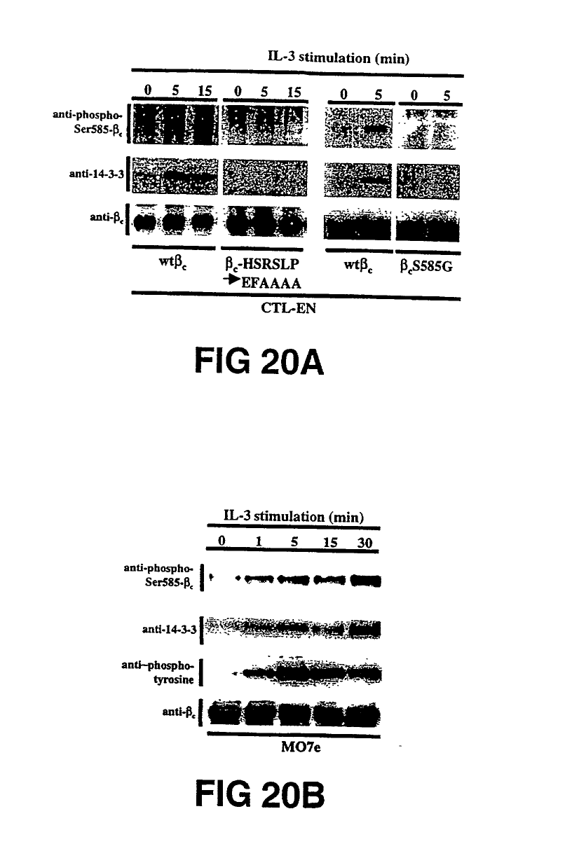

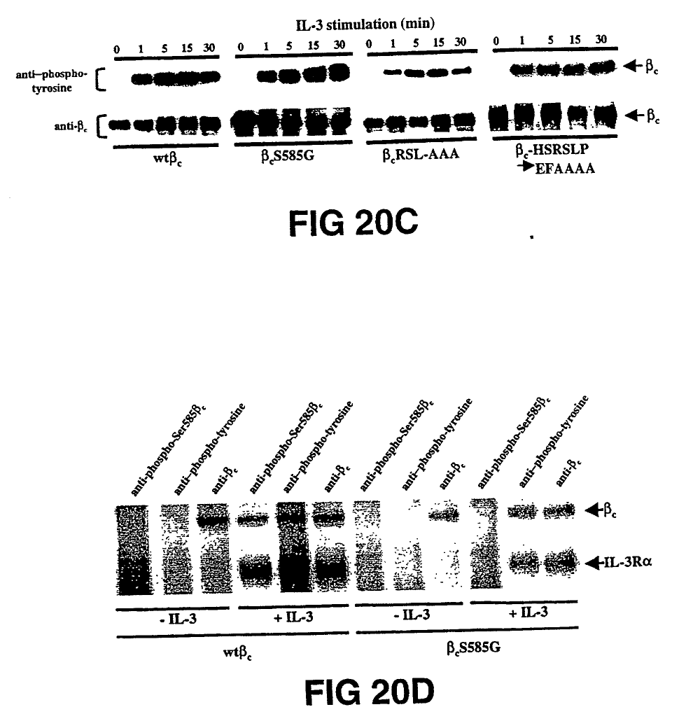

To address the functional significance of 14-3-3 binding to β_{c} cell lines were generated expressing either wild type (wt) or mutant IL-3 receptors lacking the 143-3 binding site. Constructs expressing the IL-3-specific α_{.} chain (IL-3Rα_{..} and either wt β_{c}, β_{c}HSRSLP→EFAAAA, β_{c}RSL→AAA, β_{c}S585A or β_{c}S585G were introduced into the CTL-EN T-cell line. The regulation of β_{c}Ser585 phosphorylation in response to IL-3 stimulation was examined in these cell lines using the anti-phospho-Ser585β_{c} antibody. These antibodies have been shown to specifically recognize the phosphorylated 14-3-3 binding motif HSRSLP (where S is phosphorylated) but not the non-phosphorylated motif (Stomski et al., 1999). CTL-EN cells expressing IL-3Rα and either wtβ_{c}, β_{c}HSRSLP→EFAAAA or β_{c}S585G were factor-deprived for 12 hours in 0.5% FCS and then stimulated with 50 ng/ml of IL-3. The β_{c} was immunoprecipitated and then subjected to immunoblot analysis using the anti-phospho-Ser585β_{c} antibody. Ser585 phosphorylation was upregulated in response to IL-3 stimulation in CTL-EN cells expressing wtβ_{c} while no signal was detected for either the β_{c}HSRSLP→EFAAAA or the β_{c}S585G mutant controls (FIG. 20A). Co-immunoprecipitation experiments showed that increased β_{c}Ser585 phosphorylation in response to IL-3 stimulation also resulted in increased 14-3-3 binding to β_{c} (FIG. 20A). No association of 14-3-3 with the β_{c}HSRSLP→EFAAAA or the β_{c}S585G mutant controls in response to IL-3 was observed. In addition, the MO7e cell line which expresses endogenous IL-3 receptors also demonstrated increased Ser585 phosphorylation and 14-3-3 recruitment in response to IL-3 stimulation (FIG. 20B). No difference in the kinetics or extent of tyrosine phosphorylation was observed for the β_{c}HSRSLP→EFAAAA, β_{c}RSL→AAA or the β_{c}S585G mutants when compared to the wtβ_{c} receptor (FIG. 20C). The stoichiometry of Ser585 phosphorylation in response to IL-3 in vivo was also examined and compared to β_{c} tyrosine phosphorylation. In these experiments, β_{c} was immunoprecipitated from I¹²⁵ surface-labelled CTL-EN cells (to avoid detection of intracellular-only β_{c}) with either anti-phosphorSer585, anti-phosphotyrosine or anti-β_{c} antibodies from both IL-3-stimulated (+IL-3) or non-stimulated (−IL-3) cells expressing wtβ_{c} or β_{c}S585G receptors. Immunoprecipitates were electrophoresed on a polyacrylamide gel and the amount of radiolabelled receptor immunoprecipitated was quantified using a phosphorImager. In non-stimulated cells (wtβ_{c}, β_{c} as the receptor is not phosphorylated on either Ser585 or tyrosine (FIG. 20D). Following IL-3 stimulation, similar amounts of wtβ_{c} were immunoprecipitated with the anti-phosphoSer585β_{c} and anti-phosphotyrosine antibodies when compared to the anti-β_{c} antibodies. Quantification of the bands indicated that in cells expressing the wtβ_{c}, approximately 87% of surface-labelled β_{c} is phosphorylated on Ser585 and approximately 94% of β_{c} is phosphorylated on tyrosine. While the anti-phosphoSer585β_{c} antibodies did not immunoprecipitate β_{c}S585G from IL-3 stimulated cells, both anti-phosphotyrosine and anti-β_{c} antibodies did. Approximately 95% of the surface-labelled β_{c}S585G was tyrosine phosphorylated following IL-3 stimulation which was comparable to the amount of wtβ_{c} tyrosine phosphorylation. As we have previously reported (Stomski et al., 1996), immunoprecipitation of the β_{c} from I¹²⁵ surface-labelled cells following IL-3 stimulation results in the co-immunoprecipitation of IL-3Rα. Similar levels of IL-3Rα were co-immunoprecipitated with the antiβ_{c} antibodies from cells expressing either wtβ_{c} or β_{c}S585G indicating that the receptor mutant is not impaired in its ability to dimerize with IL-3Rα in response to ligand (FIG. 20D).

### EXAMPLE 11

### cAMP-Dependent Protein Kinase (PKA) Phosphorylates Ser585 of β_{c}.

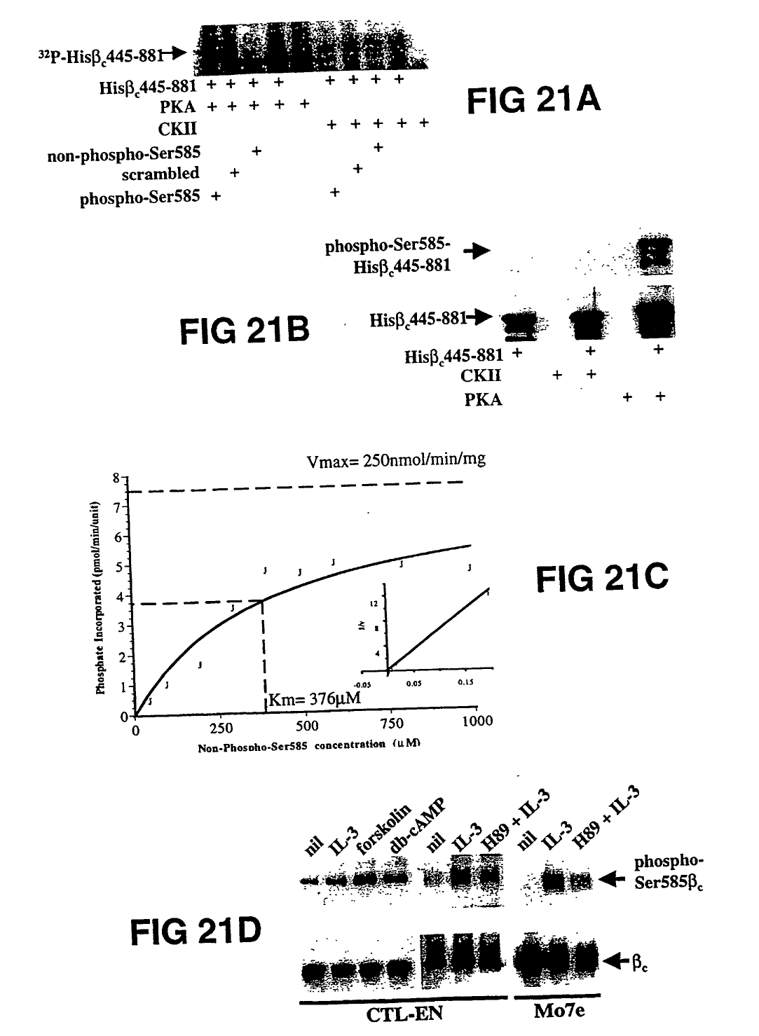

In an effort to identify the kinase involved in the phosphorylation of Ser585, the ability of several kinases to phosphorylate the recombinant intracellular portion (amino acids 445-881) of β_{c} (Hisβ_{c}445-881) in vitro was examined. Ser585 of β_{c} lies within an ⁵⁸⁵SXXD⁵⁸⁸ motif which is a possible casein kinase II (CKII) consensus phosphorylation site and also within an ⁵⁸²HSRS⁵⁸⁵ motif which is a possible, but poor, cAMP-dependent protein kinase (PKA) consensus phosphorylation site (Kennely and Krebs, (1991)) (J. Biol. Chem. 266, 15,55515,558). In the in vitro kinase assay shown in FIG. 21A, ³²P-labelling of purified Hisβ_{c}445-881 was observed for both PKA and CKII. Additional bands for the PKA kinase reactions were evident due to the autocatalytic activity of PKA. Interestingly, the presence of a non-phospho-Ser585 peptide reduced the phosphorylation of Hisβ_{c}445-881 by PKA (presumably acting as a competitive substrate) whereas a phospho-Ser585 or a scrambled peptide did not (FIG. 21A). Furthermore, while CKII can clearly phosphorylate Hisβ_{c}445-881, the phosphorylation was not inhibited by any of the peptides suggesting that CKII does not phosphorylate Ser585 (FIG. 21A).

The ability of PKA or CKII to specifically phosphorylate Ser585 of Hisβ_{c}445-881 was then examined by immunoblot analysis using the anti-phospho-Ser585β_{c} antibody. CKII was not able to detectably phosphorylate Ser585 of Hisβ_{c}445-881 whereas PKA caused strong phosphorylation of Ser585 as detected by the anti-phospho-Ser585 antibody (FIG. 21B). We then examined the affinity of PKA for the non-phospho-Ser585 peptide and the kinetics of phosphorylation. The apparent Kₘ and Vₘₐₓ for this peptide derived from hyperbolic regression analysis was 376 μM and 250 nmol/min/mg respectively (FIG. 21C).

As the results shown in FIGS. 21A-C suggested that PKA could phosphorylate Ser585 of β_{c} in vitro with Kₘ and Vₘₐₓ values within the physiological range of other known PKA substrates, we then examined the ability of PKA to phosphorylate Ser585 of β_{c} in vivo was examined. CTL-EN cells expressing the wtβ_{c} were factor-deprived for 12 hours and then either not stimulated (nil), or stimulated with 50 ng/ml IL-3, 50 μM forskolin or 100 μMdibutyryl cAMP (db-cAMP) for 10 minutes. In addition, the ability of the PKA inhibitor, H89, to block IL-3-induced phosphorylation of Ser585 of β_{c} was examined. For these experiments, either CTL-EN cells expressing the wtβ_{c} or MO7e cells were either not stimulated (nil), stimulated for 10 minutes with IL-3 (IL-3), or pretreated with 30 μM H89 for 15 minutes prior to IL-3 stimulation for 10 minutes (H89+IL-3). After various treatments, cells were lysed, β_{c} was immunoprecipitated, and subjected to immunoblot analysis with the anti-phospho-Ser585 antibody. IL-3 stimulation, as well as stimulation with agents that activate PKA (forskolin and db-cAMP), resulted in increased Ser585 phosphorylation (FIG. 21D). Furthermore, blocking PKA activation with H89 significantly reduced the IL-3 induced phosphorylation of Ser585 in both CTL-EN cells and MO7e cells.

### EXAMPLE 12

### 14-3-3 Recruitment to β_{c} in Response to IL-3 Couples the IL-3 Receptor to the PI 3-Kinase pathway.

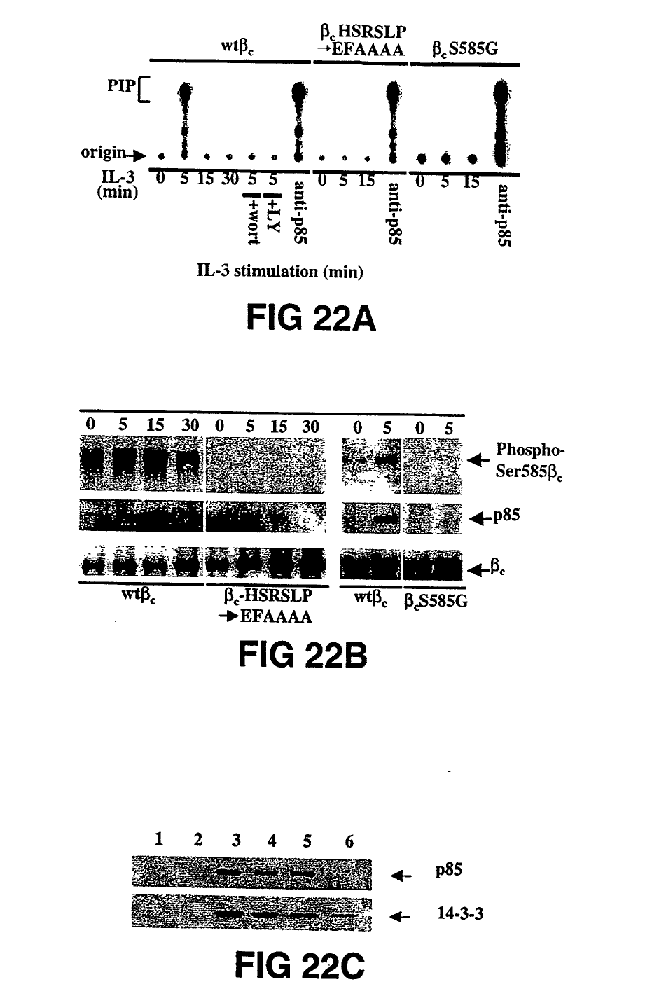

The regulation of Ser585 phosphorylation and the recruitment of 14-3-3 to β_{c} raised the possibility that these events were important in regulating IL-3 signalling. GM-CSF and IL-3 are known to regulate at least three, not necessarily mutually exclusive pathways; the JAK/STAT, the ras/MAP kinase and the PI 3-kinase pathways. The possibility that Ser585 of β_{c} and 14-3-3 association may be important in the regulation of the PI 3-kinase pathway in response to IL-3 was exmained. CTL-EN cells expressing IL-3Rα and either the wtβ_{c}, the β_{c}HSRSLP→EFAAAA mutant or the β_{c}S585G mutant were factor-deprived, stimulated with IL-3, tyrosine phosphorylated proteins were immunoprecipitated, and the PI 3-kinase activity of the immunoprecipitates was determined in an in vitro kinase assay using phosphatidylinositol and γ³²P-ATP as substrates. IL-3 stimulation of cells expressing wtβ_{c} resulted in the rapid and transient activation of PI 3-kinase activity which was blocked by the PI 3-kinase inhibitors Wortmannin and LY294002 (FIG. 22A). No PI 3-kinase activity was observed in response to IL3 stimulation for either the β_{c}HSRSLP→EFAAAA or the β_{c}S585G mutants suggesting that 14-3-3 binding is necessary for PI 3-kinase activation (FIG. 22A). As positive controls, p85 immunoprecipitates prepared from non-starved CTL-EN cells expressing either wtβ_{c}, β_{c}HSRSLP→EFAAAA or β_{c}S585G were examined and PI 3-kinase activity was clearly detectable for both cell lines.

As β_{c} does not contain a classical YXXM consensus for the direct binding of PI 3-kinase, our results raised the possibility that 14-3-3 could act as an adaptor to recruit PI 3-kinase to β_{c} via phospho-Ser585. To address this possibility we performed co-immunoprecipitation experiments examining the association of β_{c} with the p85 regulatory subunit of PI 3-kinase. CTL-EN cells expressing IL-3Rα and either wtβ_{c}, β_{c}HSRSLP→EFAAAA or β_{c}S585G were stimulated with IL-3, β_{c} was immunoprecipitated and the immunoprecipitates were examined for associated p85 subunit by immunoblot analysis. IL-3 stimulation resulted in the recruitment of p85 to wtβ_{c} whereas no recruitment of p85 to the β_{c}HSRSLP→EFAAAA or the β_{c}S585G mutants was observed (FIG. 22B).

To further examine the possibility that 14-3-3 could couple β_{c} to PI 3-kinase, we performed pull-down experiments using recombinant intracellular-portion of β_{c} (Hisβ_{c}445-881). The results presented above indicated that the association of 14-3-3 with the ⁵⁸²HSRSLP⁵⁸⁷ motif of β_{c} and the subsequent recruitment of PI 3-kinase is dependent on the phosphorylation of Ser585. We have also shown that PKA can efficiently phosphorylate Ser585 of Hisβ_{c}445-881 in vitro (FIGS. 21A, B and C). We therefore examined the ability of Hisβ_{c}445-881 coupled to Sepharose and phosphorylated on Ser585 by PKA to precipitate both p85 and 14-3-3 from COS-7 cell lysates. No interaction of either 14-3-3 or p85 was observed with Sepharose alone (FIG. 22C, lane 1) or unphosphorylated Hisβ_{c}445-881 (lane 2), however, both 14-3-3 and p85 were precipitated from COS-7 cell lysates using PKA-phosphorylated Hisβ_{c}445-881 (lane 3). A number of peptides encompassing the 14-3-3 binding site in β_{c} were examined for their ability to competitively inhibit the interaction of Hisβ_{c}445-881 with p85 and 14-3-3. Hisβ_{c}445-881 Sepharose was able to pull-down p85 and 14-3-3 in the presence of the Ser585Ala (FIG. 22C, lane 4) or non-phospho-Ser585 (lane 5) control peptides, but this interaction was markedly reduced in the presence of a phospho-Ser585 peptide (lane 6). These results further indicate that the recruitment of PI 3-kinase to β_{c} requires the presence of the HSRSLP 14-3-3-binding site in β_{c}.

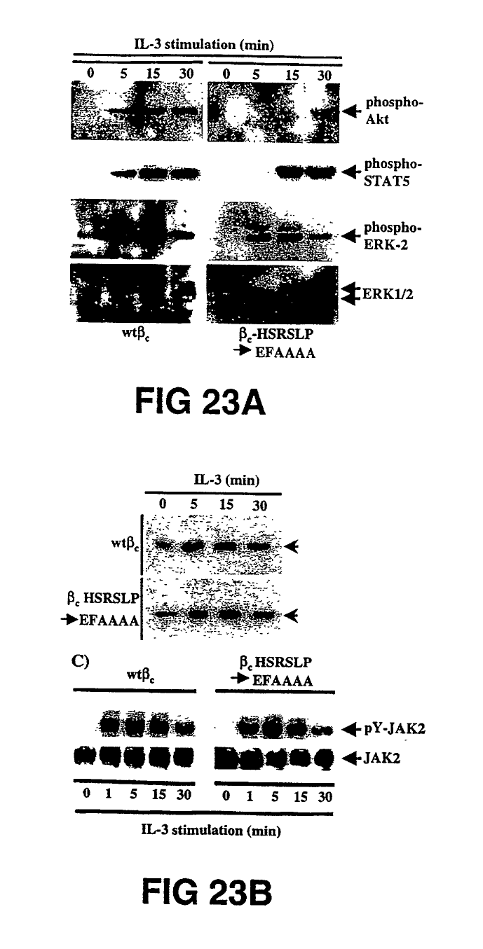

To examine whether the recruitment of 14-3-3 to β_{c} was specifically involved in signalling through the PI 3-kinase pathway, we investigated a number of additional signalling molecules known to be activated in response to IL-3. CTL-EN cells expressing either wtβ_{c} or the β_{c}HSRSLP→EFAAAA receptors were factor-deprived and then stimulated with IL-3. The phosphorylation of the signal transducer and activator of transcription protein STAT5, and the extracellular-regulated kinases ERK1 and ERK 2 in response to IL-3 was unaffected in cells expressing the mutant receptor when compared to cells expressing the wtβ_{c} (FIG. 23A). However, while CTL-EN cells expressing wtβ_{c} demonstrated phosphorylation of Akt in response to IL-3, the phosphorylation of Akt was almost completely abolished in cells expressing the β_{c}HSRSLP→EFAAAA mutant (FIG. 23A). The activation of c-jun N-terminal kinase (JNK) was also examined in JNK immunoprecipitates using an in vitro kinase assay and GST-jun as a substrate. CTL-EN cells expressing either wtβ_{c} or β_{c}HSRSLP→EFAAAA were both able to induce JNK kinase activity in response to IL-3 (FIG. 23B). Similarly, no differences in the tyrosine phosphorylation of the non-receptor tyrosine kinase, JAK2 were observed (FIG. 23C). These results show that phosphorylation of Ser585 of β_{c} and 14-3-3 association are not only important for the recruitment and activation of PI 3-kinase, but also for the activation of its downstream signalling partner Akt. Furthermore, Ser585 phosphorylation of β_{c} and 14-3-3 binding are important for the specific regulation of the PI 3-kinase pathway but not for the regulation of other known pathways emanating from β_{c} that utilize STAT5, ERK, JNK or JAK2.

### EXAMPLE 13

### Association of 14-3-3 with β_{c} is Required for IL-3-Mediated Cell Survival but not Proliferation.

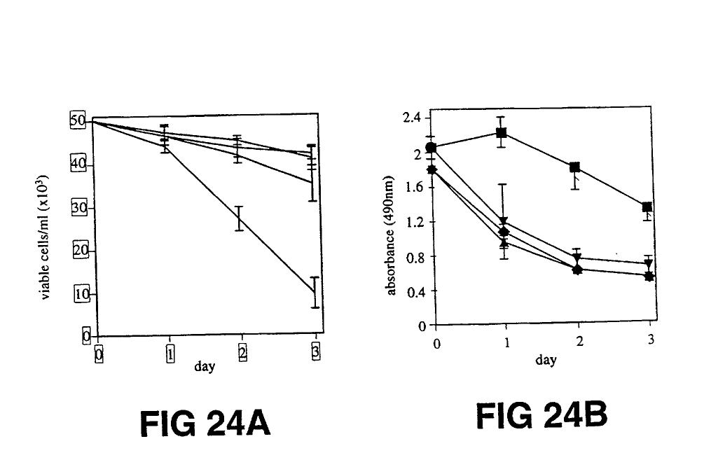

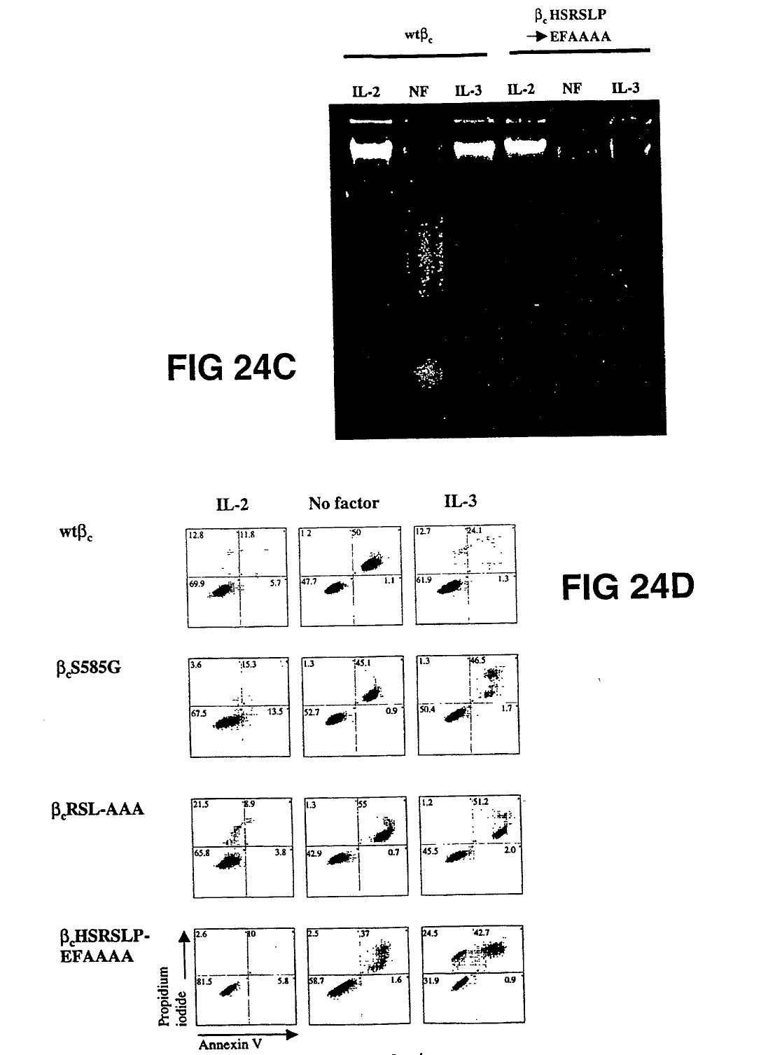

The finding that 14-3-3 binding to Ser585 of β_{c} is required for PI 3-kinase recruitment and activation in response to IL-3 and also the important role PI 3-kinase is thought to play in mediating survival signals raised the possibility that phosphorylation of Ser585 of β_{c} was important in regulating cell survival. Initial experiments performed in the presence of 10% FCS showed no defect in either the survival or growth of CTL-EN cells expressing the β_{c}HSRSLP→EFAAAA mutant receptor (data not shown). However, a defect in the survival of CTL-EN cells expressing the β_{c}HSRSLP→EFAAAA mutant was observed under low serum conditions. Thus, while serum is neither necessary nor sufficient in promoting IL-3-mediated CTL-EN cell survival (unpublished observations) it can augment IL-3-mediated survival in cells expressing the β_{c}HSRSLP→EFAAAA mutant receptor. CTL-EN cells expressing either wtβ_{c} or β_{c}HSRSLP→EFAAAA were plated out at 5×10⁵ cells/ml in the presence of 10 ng/ml IL-310.1% FCS and viable cells were counted over 3 days. While IL-3 was able to promote viability of CTL-EN cells expressing wtβ_{c} for up to 3 days, cells expressing the β_{c}HSRSLP→EFAAAA mutant showed a loss of viability in the presence of IL-3 with only 18% viable cells remaining after 3 days (FIG. 24A). The viability of CTL-EN cells expressing wtβ_{c} or β_{c}HSRSLP→EFAAAA were maintained in the presence of the IL-2 control cytokine.

Loss of cell viability in CTL-EN cells expressing the β_{c}HSRSLP→EFAAAA in response to IL-3 was also reflected in loss of cellular metabolic activity as determined by the MTS reduction assay. CTL-EN cells expressing wtβ_{c} maintain appreciable levels of metabolic activity in the presence of 10ng/ml IL-3 (▪) and 0.1% FCS for up to 3 days but lost metabolic activity in the absence of IL-3 (▾)(FIG. 24B). However, CTL-EN cells expressing the β_{c}HSRSLP→EFAAAA mutant receptor lost metabolic activity in the presence of 10 ng/ml IL-3 (FIG. 24B, ▴). To determine if the loss in cellular viability was due to apoptosis, we performed DNA laddering analysis. CTL-EN cells expressing either the wtβ_{c} or the β_{c}HSRSLP→EFAAAA mutant were plated out as described above in 20 ng/ml IL-2, no factor (NF) or 50 ng/ml IL-3. Cells were harvested 48 hours later, the DNA extracted and subjected to agarose gel electrophoresis and ethidium bromide staining. DNA from CTL-EN cells expressing either wtβ_{c} or the β_{c}HSRSLP→EFAAAA mutant exhibited the characteristic laddering typical of apoptotic cells in the absence of factor (NF) while no significant DNA laddering was observed in the presence of IL-2 (FIG. 24C). CTL-EN cells expressing wtβ_{c} and plated in IL-3 showed no significant DNA laddering, however, DNA from CTL-EN cells expressing the β_{c}HSRSLP→EFAAAA mutant which were plated in IL-3 showed a clear DNA ladder typical of apoptotic cells. As an additional means of examining the mode of cell death we performed combined annexin V and propidium iodide staining. For these experiments we examined CTL-EN cells expressing the wtβ_{c}, and the β_{c}HSRSLP→EFAAAA mutant as well as two additional mutants, β_{c}S585G and β_{c}RSL→AAA. Cells were plated out in 20 ng/ml IL-2, no factor or 50 ng/ml IL-3, stained with annexin V and propidium iodide after 30 hours and analysed by flow cytometry. In the presence of IL-2, low levels of apoptotic cells (annexin V and propidium iodide positive cells) were observed for CTL-EN cells expressing wtβ_{c} (11.8%) β_{c}S585G (15.3%), β_{c}RSL→AAA (8.9%), or β_{c}HSRSLP→EFAAAA (10.0%)(FIG. 24D). In the absence of factor, increased apoptotic cells were detected for wtβ_{c} (50.0%), β_{c}S585G (45.1%), β_{c}RSL→AAA (55.0%), and the β_{c}HSRSLP→EFAAAA (37.0%) mutant. While IL-3 was able to protect CTL-EN cells expressing wtβ_{c} against apoptosis (24.1%), cells expressing the β_{c}S585G (46.5%), β_{c}RSL→AAA (51.2%), and the β_{c}HSRSLP→EFAAAA (42.7%) mutants exhibited levels of apoptosis comparable to those observed in the absence of factor (FIG. 24D). Together, the results shown in FIGS. 24C and 24D indicate that the defect in cell survival is due to increased apoptosis.

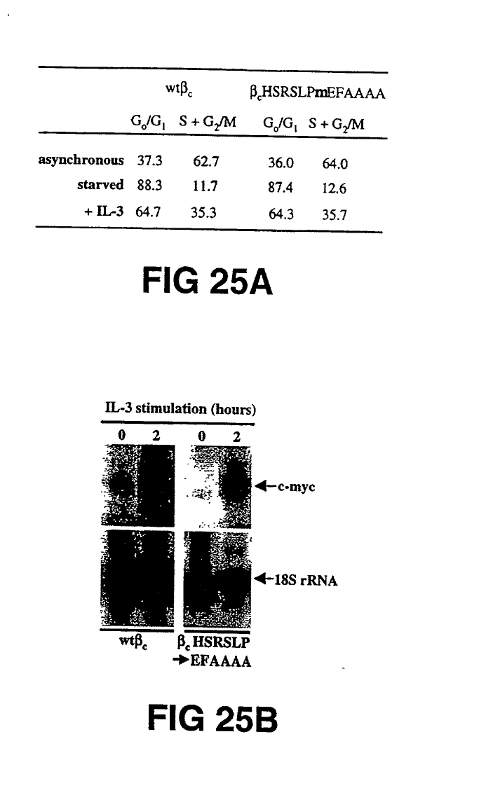

To determine if the signalling defect of the β_{c}HSRSLP→EFAAAA receptor was specifically related to cell survival or if cell proliferation was also impaired, cell cycle progression in response to IL-3 was examined. The cell cycle distribution of CTL-EN cells expressing the wtβ_{c} or the β_{c}HSRSLP→EFAAAA mutant was examined in fixed and permeablized cells by propidium iodide staining and flow cytometry. CTL-EN cells growing asynchronously in the presence of IL-2 have approximately 37% of cells in G₀/G1 phase of the cell cycle (FIG. 25A, asynchronous). Nearly 90% of CTL-EN cells accumulate in G₀/G₁ following starvation for 24 hours in DMEM containing 0.1% FCS and no cytokine (FIG. 25A, starved). IL-3 stimulation of starved cells for 24 hours resulted in equivalent numbers of CTL-EN cells expressing either wtpC or the β_{c}HSRSLP→EFAAAA mutant re-entering S phase and G₂/M phase of the cell cycle (FIG. 25A, wtβ_{c}-35.3%; β_{c}HSRSLP→EFAAAA-35.7%). These results indicate that Ser585 phosphorylation of β_{c} and 14-3-3 binding are not essential for promoting cell cycle progression. We also examined the regulation of c-myc mRNA expression in response to IL-3 by Northern blot analysis. The induction of c-myc is proposed to be an important pre-requisite for cell proliferation (Desbarats et al., 1996). No impairment of c-myc mRNA induction was observed in CTL-EN cells expressing β_{c}HSRSLP→EFAAAA when compared to control cells expressing wtβ_{c} (FIG. 25B).

### EXAMPLE 14

### Ser585 Phosphorylation and PI 3-K Signalling is Constitutive in Primary Human AML Cells.

AML cells were starved for 12 h and then stimulated with GM-CSF for 5 minutes. Where indicated cells were pre-incubated in the PKA inhibitor, H89 (10μM), for 60 min. prior to GM-CSF stimulation. Cells were then lysed. (A) 75% of the lysates were subjected to β_{c} immuno-precipitation. Immunoprecipitates were subjected to SDS-PAGE, transferred to nitrocellulose and immunoblotted with anti-phosphoSer585β_{c}. The filters were then stripped and reprobed with anti-phosphotyrosine, anti-14-3-3, anti-p85 and anti-β_{c}. (B) 25% of the lysates were subjected to anti-phosphotyrosine immuno-precipitation and PI 3-K assays were performed.

Finally it is to be understood that various other modifications and/or alterations may be made without departing from the spirit of the present invention as outlined herein.

What is claimed:

1. A binding motif of a receptor capable of binding a cytoplasmic protein, said binding motif comprising an amino acid sequence wherein at least one amino acid is serine/threonine.

2. A binding motif according to claim 1 wherein the serine/threonine residue corresponds to a serine residue at position 585 of the common β_{c} according to FIG. 1.

3. A binding motif according to claim 1 wherein at least two (2) amino acids are serine.

4. A binding motif according to claim 1 wherein the amino acid sequence includes the sequence
-S-X-S/T-
wherein the X is any amino acid.

5. A binding motif according to any claim 1 further including flanking amino acids selected from R and X-P wherein X is any amino acid such that the flanking amino acids individually or co-operatively contribute to the binding motif for binding to a cytoplasmic protein.

6. A binding motif according to claim 1 wherein the amino acid sequence of the binding motif includes the sequence:
-R-S-X-S/T-X-P-
wherein X is any amino acid.

7. A binding motif according to claim 1 wherein the the receptor is selected from the group including:
(1) GM-CSF/IL-3/IL-5 receptor
(2) IL6 human interleukin-6 receptor beta chain precursor (IL-6R-beta)
(3) LEPR human leptin receptor precursor (LEP-R) (OB RECEPTOR) (OB-R).
(4) TNR2 human tumor necrosis factor receptor 2 precursor (tumor necrosis factor
(5) VGR1 human vascular endothelial growth factor receptor 1 precursor
(6) TRK3 human receptor protein-tyrosine kinase TKT precursor (EC 2.7.1.112)
(7) Q01974 protein-tyrosine kinase transmembrane receptor ROR2 precursor
(8) FGR1 human basic fibroblast growth factor receptor 1 precursor (BFGF-R)
(9) Q15426 protein-tyrosine phosphatase, receptor-type, H precursor (EC 3.1.3.48)
(10) PTPM human protein-tyrosine phosphatase mu precursor (EC 3.1.3.48) (R-PTP-MU).
(11) PDGS human alpha platelet-derived growth factor receptor precursor (EC 2.7.1.112)
(12) FGR4 human fibroblast growth factor receptor 4 precursor (FGFR-4) (EC 2.7.1.112)
(13) FGR2 human fibroblast growth factor receptor 2 precursor (FGFR-2) (EC 2.7.1.112)
(14) Q13635 patched protein homolog (PTC)
(15) MANR human macrophage mannose receptor precursor.
(16) LRP2 human low-density lipoprotein receptor-related protein 2 precursor (megalin)
(17) IDD human integral membrane protein dgcr2/idd precursor (KIAA0163)
(18) AMFR human autocrine motility factor receptor precursor (AMF receptor) (gp78)
(19) ACH5 human neuronal acetylcholine receptor protein, alpha-5 chain precursor.
(20) KKIT human: stem cell growth factor receptor (proto-oncogene tyrosine-protein kinase kit) (C-KIT) (CD117 antigen)
(21) TPOR human: thrombopoietin receptor precusor (TPO-R) (myeloproliferative leukemia protein (C-MPL). TPOR or MPL.
(22) TPOR mouse: thrombopoietin receptor precursor (TPO-R) (myeloproliferative leukemia protein) (C-MPL). TPOR or MPL.

8. A binding motif according to claim 1 wherein the amino acid sequence is selected from the group including:
(I) HSRSLP
(ii) SSSRP
(iii) SNSKP
(iv) SDSSP
(v) SISAP
(vi) SLSLP
(vii) SASTP
(viii) SPSFP
(ix) SNSQP
(x) SVSSP
(xi) STSVP
(xii) SKSPP
(xiii) SRSQP
(xiv) SSSLP
(xv) SSSGP
(xvi) SSSFP
(xvii) SPSYP
(xviii) SGSLP
(xix) SQSSP
(xx) SPSLP
(xxi) SGSTP
(xxii) SVSPP
(xxiii) SGSGP
(xxiv) SLGSSP
(xxv) SSSQP
(xxvi) KSSERTP
(xxvii) KSSESTP
or a functional equivalent or analogue thereof.

9. A binding motif according to claim 1 wherein the receptor is the GM-CSF/IL-3/IL-5 receptor and comprises a sequence which includes amino acids HSRSLP corresponding to amino acids 582 to 587 of the common β_{c} according to FIG. 1, a functional equivalent or analogue thereof.

10. A binding motif according to claim 1 wherein the motif corresponds any one of the following:
Stem Cell Growth Factor Receptor (C-Kit) (Proto-Oncogene Tyrosine-Protein Kinase Kit) (C-KIT) (CD 117 Antigen), including amino acids 863 to 869 according to FIG. 2 or amino acid residues 965 to 969 according to FIG. 2 or a functional equivalent or analogue thereof;
Thrombopoietin Receptor Precursor (TPO-R) (Myeloproliferative Leukemia Protein) (C-MPL), (TPOR or MPL) including amino acids 573 to 579 according to FIG. 3 or a functional equivalent or analogue thereof;
Thrombopoietin Receptor Precursor (TPO-R) (Myeloproliferative Leukemia Protein) (C-MPL), (TPOR or MPL) including amino acids 564 to 570 according to FIG. 4 or a functional equivalent or analogue thereof;
IL6B HUMAN interleukin-6 receptor beta chain precursor (IL-6R-BETA), including amino acids 735-739 having the sequence SSSRP or a functional equivalent or analogue thereof;
LEPR HUMAN leptin receptor precursor (LEP-R) (OB receptor) (OB-R), including amino acids 991-995 having the sequence SNSKP or a functional equivalent or analogue thereof;
TNR2 HUMAN tumor necrosis factor receptor 2 precursor (tumor necrosis factor) including amino acids 368-372 having the sequence SDSSP or a functional equivalent or analogue thereof;
VGR1 HUMAN vascular endothelial growth factor receptor 1 precursor, including amino acids 1197-1201 having the sequence SISAP or a functional equivalent or analogue thereof;
TRK3 HUMAN receptor protein-tyrosine kinase TKT precursor (EC 2.7.1.112), including amino acids 444-448, having the sequence SLSLP or a functional equivalent or analogue thereof;
Q01974 protein-tyrosine kinase transmembrane receptor ROR2 precursor, including amino acids 435-439, having the sequence SASTP or a functional equivalent or analogue thereof;
FGR1 HUMAN basic fibroblast growth factor receptor 1 precursor (BFGF-R), including amino acids 777-781, having the sequence SPSFP or a functional equivalent or analogue thereof;
Q15426 protein-tyrosine phosphatase, receptor-type, H precursor (EC 3.1.3.48), including amino acids 1082-1086, having the sequence SNSQP or a functional equivalent or analogue thereof;
PTPM HUMAN protein-tyrosine phosphatase MU precursor (EC 3.1.3.48) (RPTP-MU), including amino acids 818-822, 833-837, 1082-1086 having the sequences SVSSP, STSVP, SKSPP or functional equivalents or analogues thereof;
PGDS HUMAN alpha platelet-derived growth factor receptor precursor (EC 2.7.1.112), including amino acids 616-620 having the sequence SRSQP or a functional equivalent or analogue thereof;
FGR4 HUMAN fibroblast growth factor receptor 4 precursor (FGFR-4) (EC 2.7.1.112), including amino acids 439-443, 791-795 having the sequences SSSGP, SSSFP or functional equivalents or analogues thereof;
FGR2 HUMAN fibroblast growth factor receptor 2 precursor (FGFR-2) (EC 2.7.1.112), including amino acids 780-784 having the sequence SPSYP or a functional equivalent or analogue thereof;
Q13635 patched protein homolog (PTC), including amino acids 1290-1294 having the sequence SGSLP or a functional equivalent or analogue thereof;
MANR HUMAN macrophage mannose receptor precursor, including amino acids 1432-1436 having the sequence SQSSP or a functional equivalent or analogue thereof;
LRP2 HUMAN low-density lipoprotein receptor-related protein 2 precursor (megalin), including amino acids 4616-4620 having the sequence SPSLP or a functional equivalent or analogue thereof;
IDD HUMAN integral membrane protein DGCR2/IDD precursor (KIAA0163), including amino acids 526-530 having the sequence SGSTP or a functional equivalent or analogue thereof;
AMFR HUMAN autocrine motility factor receptor precursor (AMF receptor) (GP78), including amino acids 203-207 having the sequence SVSPP or a functional equivalent or analogue thereof; and
ACH5 HUMAN neuronal acetylcholine receptor protein, alpha-5 chain precursor, including amino acids 382-386 having the sequence SGSGP or a functional equivalent or analogue thereof.

11. A binding motif according to claim 1 wherein at least one serine residue of the amino acid sequence is phosphorylated.

12. A binding motif according to claim 11 wherein a second serine residue from the 5′ end of the motif is phosphorylated.

13. A binding motif according to claim 11 wherein the receptor is a GM-CSF/IL-3/IL-5 receptor capable of binding a cytoplasmic protein, said binding motif comprising an amino acid sequence including the sequence ⁵⁸²HSRSLP⁵⁸⁷ of the GM-CSF/IL-3/IL-5 receptor or a functional equivalent or analogue thereof wherein at least Ser⁵⁸⁵ is capable of being phosphorylated.

14. A phophorylated binding motif of a GM-CSF/IL-3/IL-5 receptor capable of binding a cytoplasmic protein, said binding motif comprising an amino acid sequence including the sequence ⁵⁸²HSRSLP⁵⁸⁷ of the GM-CSF/IL-3/IL-5 receptor or a functional equivalent or analogue thereof wherein at least Ser⁵⁸⁵ is phosphorylated.

15. A method of phosphorylating a binding motif of a receptor capable of binding a cytoplasmic protein, said binding motif comprising an amino acid sequence, a functional equivalent or analogue thereof and wherein at least one amino acid is serine, said method comprising binding a triggering molecule to the receptor.

16. A method according to claim 15 wherein the serine residue corresponds to a serine residue at position 585 of the common β_{c} according to FIG. 1.

17. A method according to claim 15 wherein the triggering molecule corresponds to its receptor.

18. A method according to claim 17 wherein the triggering molecule is a cytokine which binds to its corresponding receptor.

19. A method according to claim 18 wherein the receptor is a GM-CSF/IL-5/IL-3 and the triggering molecule is selected from the group including GM-CSF, IL-5 or IL-3.

20. A method of binding a cytoplasmic protein to a receptor, said method comprising:
phosphorylating a binding motif according to claim 1, a functional equivalent or analogue thereof; and
subjecting the binding motif of the receptor to a cytoplasmic protein.

21. A method of activating cellular activities said method including:
regulating the activation of phosphorylation of a binding motif of a receptor according to claim 1, a functional equivalent or analogue thereof; and
subjecting the binding motif to a cytoplasmic protein wherein said cytoplasmic protein is associated with cellular activities.

22. A method of regulating cellular activities, said method including:
regulating the phosphorylation of a binding motif of a receptor according to claim 1, a functional equivalent or analogue thereof;
subjecting the binding motif to a cytoplasmic protein to bind the cytoplasmic protein to the binding motif; and
activating a cell signaling pathway by interacting the bound cytoplasmic protein with a signaling molecule involved in the pathway.

23. A method according to claim 20 wherein the cytoplasmic protein is 14-3-3 protein.

24. A method according to claim 21 wherein the cellular activities or cell signalling pathways are selected from the group including cell survival, proliferation, transformation, differentiation, mitogenesis, chemotaxis, motility, enhanced phagocytosis, bacterial killing, superoxide production and cytotoxicity.

25. A method according to claim 23 wherein the cell signaling pathway is the PI-3-kinase pathway.

26. A method of inhibiting cell survival, said method including inhibiting the binding of a cytoplasmic protein to a binding motif of a receptor according to claim 1.

27. A method according to claim 26 wherein inhibition of the binding is via an antagonist which binds to the receptor motif, said antagonist selected from the group including antibodies, small peptides, small molecules, peptide mimetics or any type of molecule known to those skilled in the art that are directed to the cytoplasmic binding motif so as to prevent attachment of cytoplasmic proteins to a phosphoserine residue or a signaling molecule.

28. A method of inhibiting cell activation, said method including inhibiting the binding of a cytoplasmic protein to a binding motif of a receptor according to claim 1.

29. A method of treating a cytokine mediated condition said method comprising:
regulating the activation of phosphorylation of a binding motif of a receptor according to claim 1, a functional equivalent or analogue thereof.
